(19) United States
(12) Patent Application Publication
(10) Pub. No.: US 2025/0051290 A1
(43) Pub. Date: Feb. 13, 2025

(54) NOVEL COMPOUND FOR IMPROVING SLEEP OR USE THEREOF

(71) Applicants: Ajou University Industry-Academic Cooperation Foundation, Suwon-si, Gyeonggi-do (KR); Sangmyung University Cheonan Council for Industry-Academic Cooperation Foundation, Cheonan-si, Chungcheongnam-do (KR)

(72) Inventors: Yi-Sook Jung, Yongin-si, Gyeonggi-do (KR); Eunha Kim, Seoul (KR); Sunghou Lee, Yongin-si, Gyeonggi-do (KR); Hye Jin Jee, Hwaseong-si, Gyeonggi-do (KR); Ju-Young Park, Suwon-si, Gyeonggi-di (KR)

(21) Appl. No.: 18/723,115

(22) PCT Filed: Dec. 26, 2022
(86) PCT No.: PCT/KR2022/021333

§ 371 Date: Oct. 15, 2024

(30) Foreign Application Priority Data
Dec. 24, 2021 (KR) 10-2021-0187788

(51) Int. Cl. C07D 239/95; A61K 31/517; A61P 25/20; C07D 403/04; C07D 403/14; C07D 405/12; C07D 405/14

(52) U.S. Cl. CPC C07D 239/95; A61K 31/517; A61P 25/20; C07D 403/04; C07D 403/14; C07D 405/12; C07D 405/14

(57) ABSTRACT

The present invention relates to a novel compound which can provide a sleep improving effect or the use thereof. Some compounds have an adenosine receptor agonistic activity. The novel compound of the present invention can shorten sleep latency and extend total sleep duration, and thus may be effectively used for a pharmaceutical composition for preventing or treating a sleep disorder, a health functional food composition for preventing or relieving a sleep disorder, a composition for improving sleep, a sleep aid, or a method for treating a sleep disorder.

### TECHNICAL FIELD

The present application claims priority to Korea Patent Application No. 10-2021-0187788 filed on Dec. 24, 2021, and the entire specification is hereby incorporated by reference into the present application.

The present invention relates to a novel compound that can provide a sleep improvement effect and a use thereof.

The present invention was completed under the project number 2020-JDH-2-CG-1 (1711124481) with the support of the Ministry of Science and ICT and the Gyeonggi-do Regional Research Center (GRRC) project of Gyeonggi-do (GRRCAjou2023-B01) of the Republic of Korea.

### BACKGROUND ART

Sleep refers to a state in which conscious activity rests with the eyes closed. This is an important process in which people replenish the energy consumed during daytime activities and recover from fatigue accumulated due to physical activity, and is also the time when growth hormones, which are essential for human growth, are secreted in the greatest amount. In addition, rest is necessary for the brain, which oversees all physiological functions to maintain life in our body, to maintain an appropriate balance of activity, and such rest mostly occurs during sleep. Recently, the American Thoracic Society recommended that adults should sleep six to nine hours a day.

However, the number of patients receiving treatment for sleep problems has increased in recent years due to the exhausting and busy daily lives of modern people and the aging of the population and is expected to continue to increase in the future. Sleep-related disorders directly damage health, and recent studies show that lack of sleep increases the risk of diabetes, heart disease, and obesity. In a study in the journal ‘Sleep’ published in 2004, women who slept less than five hours at night on average had a significantly higher mortality rate than women who slept seven hours.

Most of the research and development of therapeutics for mental disorders such as anxiety and sleep disorders caused by stress is focused on the gamma-aminobutyric acid (GABA) nervous system, serotonin nervous system, noradrenaline nervous system, and neuropeptide system, so many therapeutics developed so far have many limitations on their use due to severe side effects.

### RELATED ART DOCUMENT

### Patent Document

KR 10-2012-7028611 (Mar. 29, 2011)

### DISCLOSURE

### Technical Problem

Accordingly, the present inventors made diligent efforts to provide a novel compound that has a sleep improvement effect, and as a result, they confirmed that 28 compounds can provide a sleep improvement effect, thereby completing the present invention.

### Technical Solution

The present invention provides a pharmaceutical composition for preventing or treating a sleep disorder, including a compound represented by [Chemical Formula 1] below, an isomer thereof, or a pharmaceutically acceptable salt thereof:

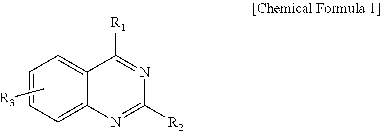

wherein R₁ or R₂ is any one selected from the group consisting of hydrogen, a straight-chain or branched C₁-C₃ alkyl, a hydrogen,

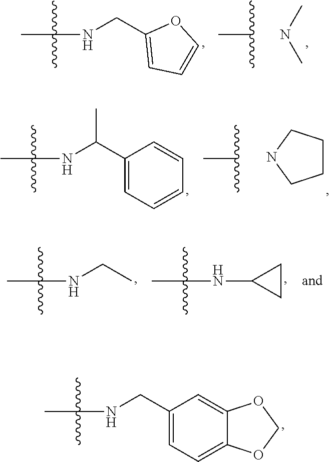

and
R₃ is one or more selected from the group consisting of hydrogen, a straight-chain or branched C₁-C₃ alkyl, a halogen, hydroxy, and methoxy.

According to one preferred embodiment of the present invention, R₁ is any one selected from the group consisting of

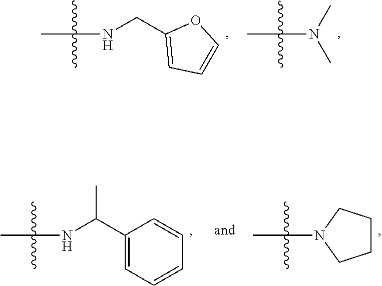

R₂ is any one selected from the group consisting of

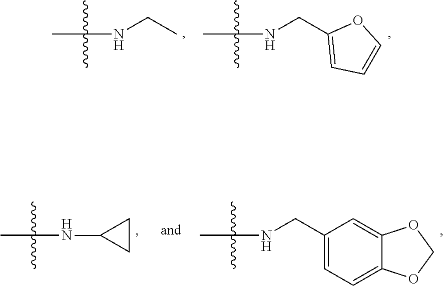

and
R₃ is one or more selected from the group consisting of hydrogen, hydroxy, and methoxy.

According to one preferred embodiment of the present invention, the sleep disorder is one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

In addition, the present invention provides a health functional food composition for preventing or relieving a sleep disorder, including a compound represented by [Chemical Formula 1] below, an isomer thereof, or a pharmaceutically acceptable salt thereof.

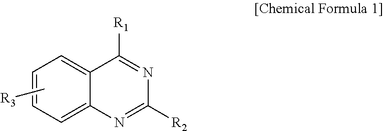

wherein R₁ or R₂ is any one selected from the group consisting of hydrogen, a straight-chain or branched C₁-C₃ alkyl, a halogen,

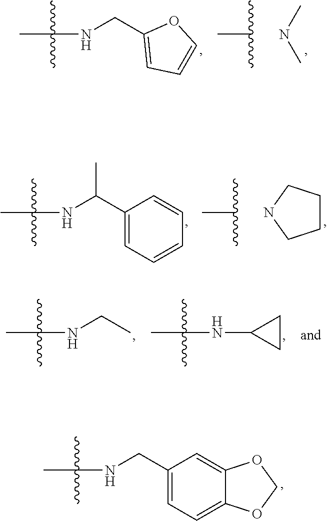

and
R₃ is one or more selected from the group consisting of hydrogen, a straight-chain or branched C₁-C₃ alkyl, a halogen, hydroxy, and methoxy.

According to one preferred embodiment of the present invention, the sleep disorder is one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

In addition, the present invention provides a composition for sleep improvement, including a compound represented by [Chemical Formula 1] below, an isomer thereof, or a pharmaceutically acceptable salt thereof.

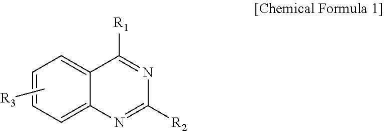

wherein R₁ or R₂ is any one selected from the group consisting of hydrogen, a straight-chain or branched C₁-C₃ alkyl, a halogen,

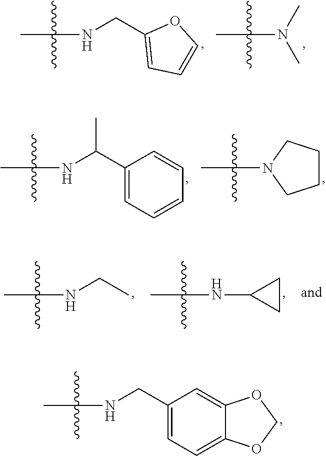

and
R₃ is one or more selected from the group consisting of hydrogen, a straight-chain or branched C₁-C₃ alkyl, a halogen, hydroxy, and methoxy.

In addition, the present invention provides a sleep aid including a compound represented by [Chemical Formula 1] below, an isomer thereof, or a pharmaceutically acceptable salt thereof:

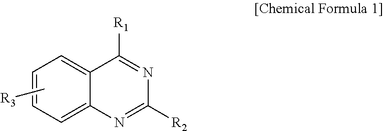

wherein R₁ or R₂ is any one selected from the group consisting of hydrogen, straight-chain or branched C₁-C₃ alkyl, a halogen,

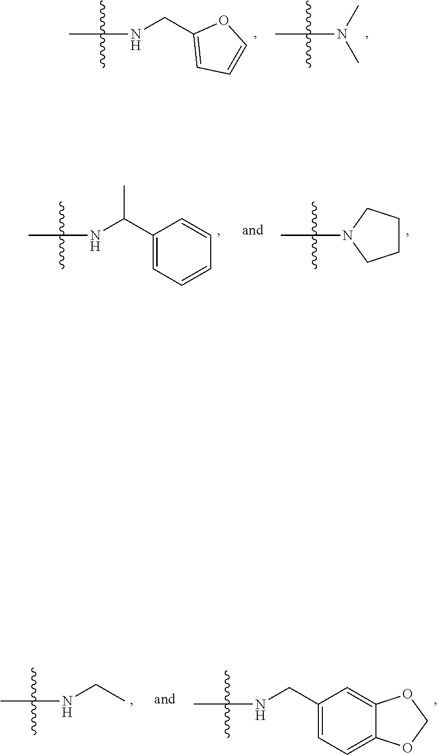

and
R₃ is one or more selected from the group consisting of hydrogen, straight-chain or branched C₁-C₃ alkyl, a halogen, hydroxy, and methoxy.

In addition, the present invention provides a compound represented by [Chemical Formula 1] below, an isomer thereof, or a pharmaceutically acceptable salt thereof:

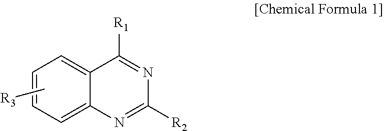

wherein R₁ is any one selected from the group consisting of

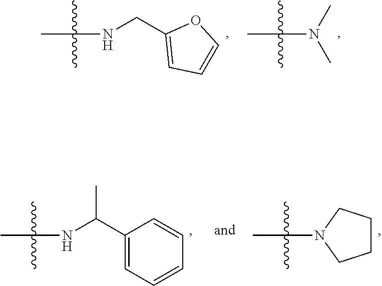

R₂ is any one selected from the group consisting

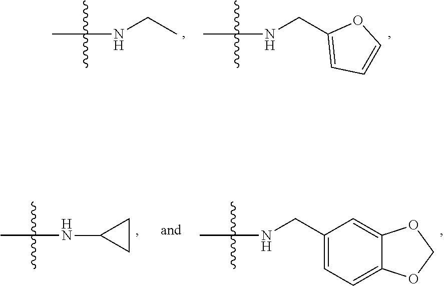

and
R₃ is one or more selected from the group consisting of hydrogen, hydroxy, and methoxy.

In addition, the present invention provides a method of treating a sleep disorder, including administering a compound represented by [Chemical Formula 1] below, an isomer thereof, or a pharmaceutically acceptable salt thereof to a patient with a sleep disorder,

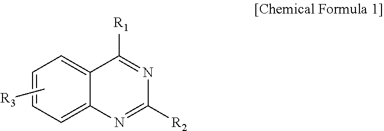

wherein R₁ or R₂ is any one selected from the group consisting of hydrogen, a straight-chain or branched C₁-C₃ alkyl, a halogen,

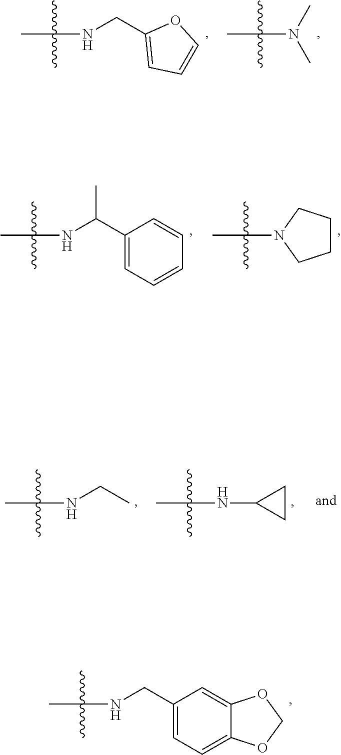

and
R₃ is one or more selected from the group consisting of hydrogen, a straight-chain or branched C₁-C₃ alkyl, a halogen, hydroxy, and methoxy.

According to one preferred embodiment of the present invention, the sleep disorder is one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

In addition, the present invention provides a use of a compound represented by [Chemical Formula 1] below, an isomer thereof, or a pharmaceutically acceptable salt thereof for treatment of a sleep disorder:

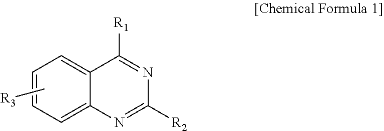

wherein R₁ or R₂ is any one selected from the group consisting of hydrogen, a straight-chain or branched C₁-C₃ alkyl, a halogen,

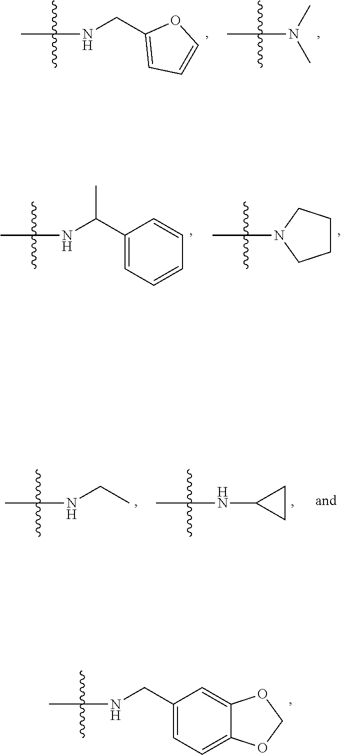

and
R₃ is one or more selected from the group consisting of hydrogen, a straight-chain or branched C₁-C₃ alkyl, a halogen, hydroxy, and methoxy.

According to one preferred embodiment of the present invention, the sleep disorder is one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

To discover compounds that can provide a sleep improvement effect, the present inventors conducted research at the Korea Chemical Bank of the Korea Research Institute of Chemical Technology and as a result, they synthesized and discovered a total of 28 compounds with a sleep improvement effect.

Specifically, it was intended to synthesize quinazoline analogs with various amine substituents through the reaction scheme of FIG. 1. Reaction (a) involved substituting urea with dichloroquinazoline 2 using POCl₃ as a solvent, but the yield was not high, so reagents were purchased and the next reaction was performed. Through Reaction (b), a chloride group at a benzylic position of dichloroquinazoline was substituted with an amine, and when using sodium acetate as a base and using tetrahydrofuran (TIF) and water under 3/1 conditions, the desired compounds (3a to 3d) were obtained. Reaction (c) was to substitute the remaining chloride group with an amine, and due to the low reactivity, the reaction was carried out in a sealed tube by raising the temperature of the ethanol solvent to 150° C. Since a large amount of by-product was generated when the amine was increased to three equivalents, the reaction was carried out by adding one equivalent. After the reaction was completed, the solvent was removed by distillation under reduced pressure, and the resulting solid was recrystallized with ethyl acetate, methanol, or methylene chloride to obtain 28 novel compounds (4a to 4x) and (5a to 5d).

Therefore, the present invention may provide a pharmaceutical composition for preventing or treating a sleep disorder, including a compound represented by [Chemical Formula 1] below, an isomer thereof, or a pharmaceutically acceptable salt thereof:

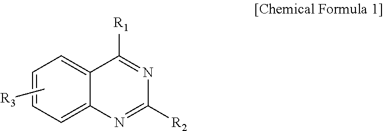

wherein R₁ or R₂ is any one selected from the group consisting of hydrogen, a straight-chain or branched C₁-C₃ alkyl, a halogen,

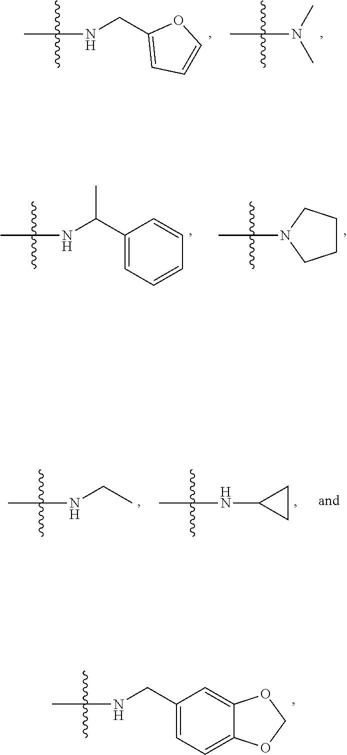

and
R₃ is one or more selected from the group consisting of hydrogen, a straight-chain or branched C₁-C₃ alkyl, a halogen, hydroxy, and methoxy.

According to one preferred embodiment of the present invention, R₁ may be any one selected from the group consisting of,

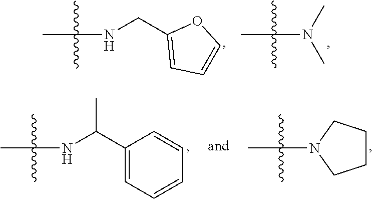

R₂ may be any one selected from the group consisting of

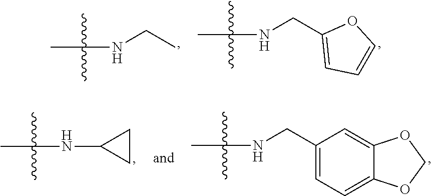

and R₃ may be one or more selected from the group consisting of hydrogen, hydroxy, and methoxy.

Preferably, the compound represented by [Chemical Formula 1] may be:

### (1) N²-ethyl-N⁴-(furan-2-ylmethyl)quinazoline-2,4-diamine (4a, AR-01, Chemical Formula 2)

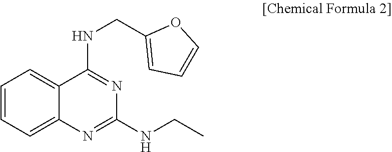 [SMILES: CCNc1nc(NCc2ccco2)c2ccccc2n1]

### (2) N²-(furan-2-ylmethyl)-N⁴,N⁴-dimethylquinazoline-2,4-diamine (4b, AR-02, Chemical Formula 3)

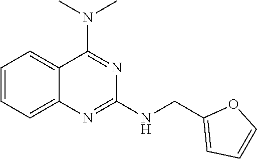 [SMILES: CN(C)c1nc(NCc2ccco2)nc2ccccc12]

### (3) N²-cyclopropyl-N⁴-(1-phenylethyl)quinazoline-2,4-diamine (4c, AR-03, Chemical Formula 4)

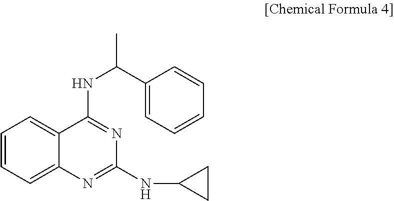 [SMILES: CC(Nc1nc(NC2CC2)nc2ccccc12)c1ccccc1]

### (4)N-(benzo[d][1,3]dioxol-5-ylmethyl)-4-(pyrrolidin-1-yl)quinazolin-2-amine (4d, AR-04, Chemical Formula 5)

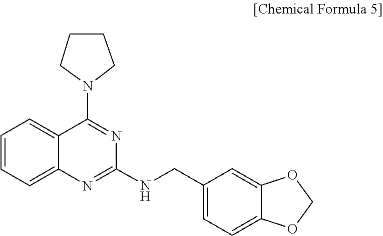 [SMILES: c1ccc2c(N3CCCC3)nc(NCc3ccc4c(c3)OCO4)nc2c1]

### (5) N²,N⁴-bis(furan-2-ylmethyl)quinazoline-2,4-diamine (4e, AR-05, Chemical Formula 6)

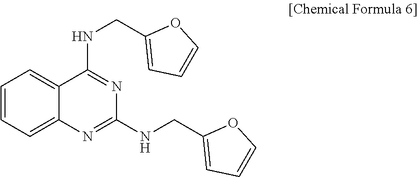 [SMILES: c1coc(CNc2nc(NCc3ccco3)c3ccccc3n2)c1]

### (6) N²-cyclopropyl-N⁴-(furan-2-ylmethyl)quinazoline-2,4-diamine (4f, AR-06, Chemical Formula 7)

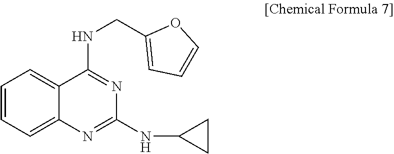 [SMILES: c1coc(CNc2nc(NC3CC3)nc3ccccc23)c1]

### (7) N²-(benzo[d][1,3]dioxol-5-ylmethyl)-N⁴-(furan-2-ylmethyl)quinazoline-2,4-diamine (4 g, AR-07, Chemical Formula 8)

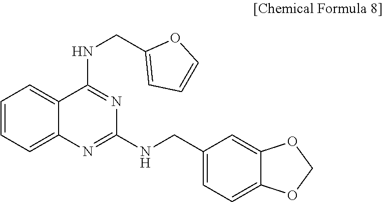 [SMILES: c1coc(CNc2nc(NCc3ccc4c(c3)OCO4)nc3ccccc23)c1]

### (8) N²-ethyl-N⁴,N⁴-dimethylquinazoline-2,4-diamine (4 h, AR-08, Chemical Formula 9)

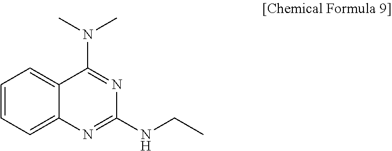 [SMILES: CCNc1nc(N(C)C)c2ccccc2n1]

### (9) N²-cyclopropyl-N⁴,N⁴-dimethylquinazoline-2,4-diamine (4i, AR-09, Chemical Formula 10)

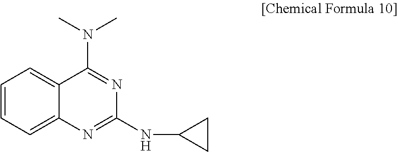 [SMILES: CN(C)c1nc(NC2CC2)nc2ccccc12]

### (10) N²-(benzo[d][1,3]dioxol-5-ylmethyl)-N⁴,N⁴-dimethylquinazoline-2,4-diamine (4j, AR-10, Chemical Formula 11)

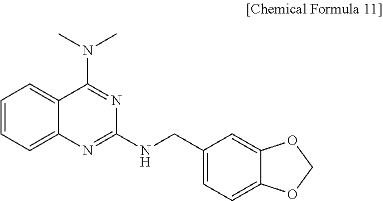 [SMILES: CN(C)c1nc(NCc2ccc3c(c2)OCO3)nc2ccccc12]

### (11) N²-ethyl-N⁴-(1-phenylethyl)quinazoline-2,4-diamine (4k, AR-11, Chemical Formula 12)

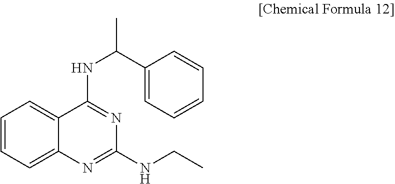 [SMILES: CCNc1nc(NC(C)c2ccccc2)c2ccccc2n1]

### (12) N²-(furan-2-ylmethyl)-N⁴-(1-phenylethyl)quinazoline-2,4-diamine (4I, AR-12, Chemical Formula 13)

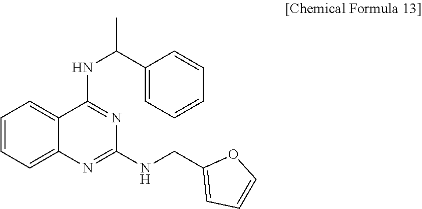 [SMILES: CC(Nc1nc(NCc2ccco2)nc2ccccc12)c1ccccc1]

### (13) N²-(benzo[d][1,3]dioxol-5-ylmethyl)-N⁴-(1-phenylethyl)quinazoline-2,4-diamine (4m, AR-13, Chemical Formula 14)

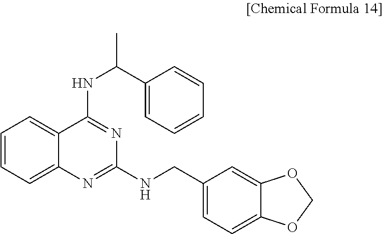 [SMILES: CC(Nc1nc(NCc2ccc3c(c2)OCO3)nc2ccccc12)c1ccccc1]

### (14)N-ethyl-4-(pyrrolidin-1-yl)quinazolin-2-amine (4n, AR-14, Chemical Formula 15)

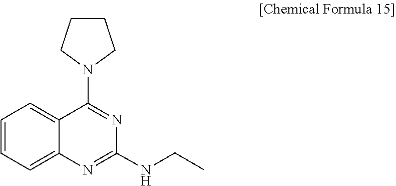 [SMILES: CCNc1nc(N2CCCC2)c2ccccc2n1]

### (15)N-(furan-2-ylmethyl)-4-(pyrrolidin-1-yl)quinazolin-2-amine (4o, AR-15, Chemical Formula 16)

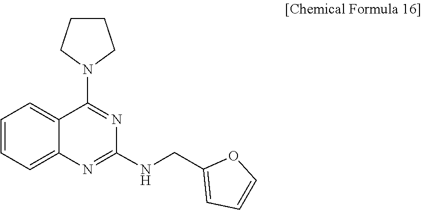 [SMILES: c1coc(CNc2nc(N3CCCC3)c3ccccc3n2)c1]

### (16)N-cyclopropyl-4-(pyrrolidin-1-yl)quinazolin-2-amine (4p, AR-16, Chemical Formula 17)

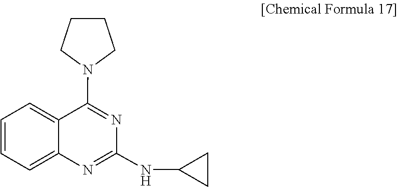 [SMILES: c1ccc2c(N3CCCC3)nc(NC3CC3)nc2c1]

### (17)N-cyclopropyl-6,7-dimethoxy-4-(pyrrolidin-1-yl)quinazolin-2-amine, 4u, AR-17, Chemical Formula 18)

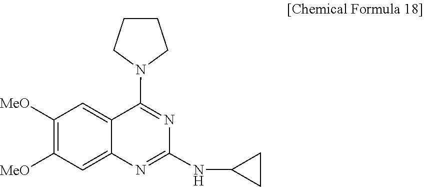 [SMILES: COc1cc2nc(NC3CC3)nc(N3CCCC3)c2cc1OC]

### (18) N²-ethyl-N⁴-(furan-2-ylmethyl)-6,7-dimethoxyquinazoline-2,4-diamine (4v, AR-18, Chemical Formula 19)

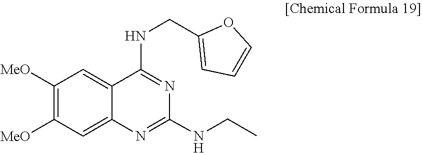 [SMILES: CCNc1nc(NCc2ccco2)c2cc(OC)c(OC)cc2n1]

### (19) N²-(furan-2-ylmethyl)-6,7-dimethoxy-N⁴,N⁴-dimethylquinazoline-2,4-diamine (4w, AR-19, Chemical Formula 20)

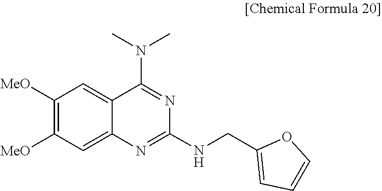 [SMILES: COc1cc2nc(NCc3ccco3)nc(N(C)C)c2cc1OC]

### (20)N-ethyl-6,7-dimethoxy-4-(pyrrolidin-1-yl)quinazolin-2-amine (4x, AR-20, Chemical Formula 21)

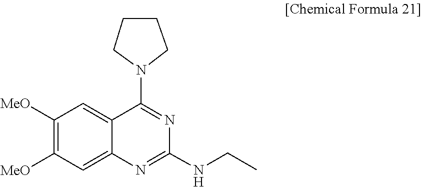 [SMILES: CCNc1nc(N2CCCC2)c2cc(OC)c(OC)cc2n1]

### (21) 2,4-di(pyrrolidin-1-yl)quinazoline (4q, AR-21, Chemical Formula 22)

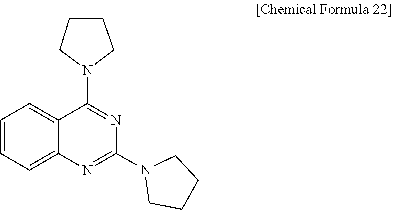 [SMILES: c1ccc2c(N3CCCC3)nc(N3CCCC3)nc2c1]

### (22) N,N-dimethyl-4-(pyrrolidin-1-yl)quinazolin-2-amine (4r, AR-22, Chemical Formula 23)

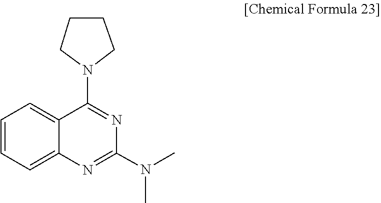 [SMILES: CN(C)c1nc(N2CCCC2)c2ccccc2n1]

### (23)N-(1-phenylethyl)-4-(pyrrolidin-1-yl)quinazolin-2-amine (4s, AR-23, Chemical Formula 24)

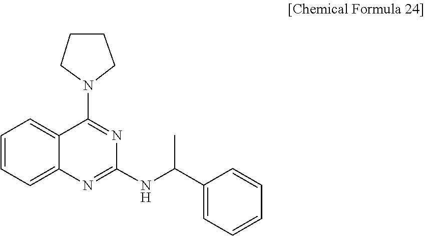 [SMILES: CC(Nc1nc(N2CCCC2)c2ccccc2n1)c1ccccc1]

### (24) 3,4-Dihydroxy-N-(2-((4-(pyrrolidin-1-yl)quinazolin-2-yl)amino) ethyl)benzamide (4t, AR-24, Chemical Formula 25)

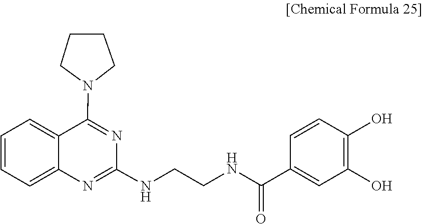 [SMILES: O=C(NCCNc1nc(N2CCCC2)c2ccccc2n1)c1ccc(O)c(O)c1]

### (25) 2-(Cyclopropylamino)-4-(pyrrolidin-1-yl)quinazoline-6,7-diol (5a, AR-25, Chemical Formula 26) [Chemical Formula 26]

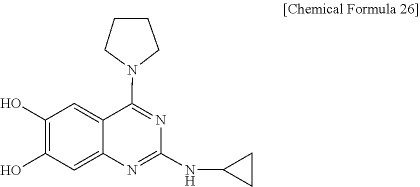 [SMILES: Oc1cc2nc(NC3CC3)nc(N3CCCC3)c2cc1O]

### (26) 4-(Dimethylamino)-2-((furan-2-ylmethyl)amino)quinazoline-6,7-diol (5b, AR-26, Chemical Formula 27)

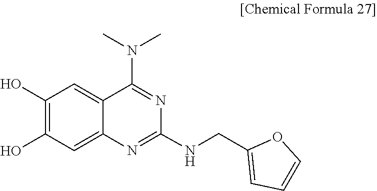 [SMILES: CN(C)c1nc(NCc2ccco2)nc2cc(O)c(O)cc12]

### (27) 2-(Ethylamino)-4-(pyrrolidin-1-yl)quinazoline-6,7-diol (5c, AR-27, Chemical Formula 28)

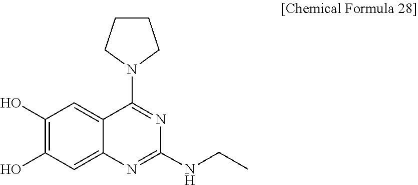 [SMILES: CCNc1nc(N2CCCC2)c2cc(O)c(O)cc2n1]

### (28) 2-(Ethylamino)-4-((furan-2-ylmethyl)amino)quinazoline-6,7-diol (5d, AR-28, Chemical Formula 29)

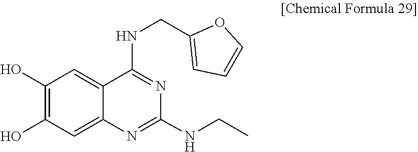 [SMILES: CCNc1nc(NCc2ccco2)c2cc(O)c(O)cc2n1]

The isomer may be any one or more selected from the group consisting of structural isomers, enantiomers, optical isomers, stereoisomers, and diastereomers.

As the pharmaceutically acceptable salt, an acid addition salt formed by a pharmaceutically acceptable free acid is useful. An acid addition salt is obtained from inorganic acids such as hydrochloric acid, nitric acid, phosphoric acid, sulfuric acid, hydrobromic acid, hydroiodic acid, nitrous acid or phosphorous acid, and non-toxic organic acids such as aliphatic mono- and dicarboxylates, phenyl-substituted alkanoates, hydroxyalkanoates and alkanedioates, aromatic acids, and aliphatic and aromatic sulfonic acids. These pharmaceutically non-toxic salts include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methyl benzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, O-hydroxybutyrate, glycolate, malate, tartrate, methanesulfonate, propanesulfonate, naphthalene-1-sulfonate, naphthalene-2-sulfonate or mandelate.

An acid addition salt according to the present invention may be prepared by a conventional method, for example, by dissolving a compound represented by [Chemical Formula 1] in an aqueous solution of excess acid and precipitating the resulting salt in a water-miscible organic solvent such as methanol, ethanol, acetone, or acetonitrile. The acid addition salt may also be prepared by evaporating the solvent or the excess acid from this mixture and then drying the precipitated salt or by suction-filtering the precipitated salt.

In addition, a pharmaceutically acceptable metal salt may also be prepared using a base. An alkali metal or alkaline earth metal salt is obtained, for example, by dissolving a compound in a solution of excess alkali metal hydroxide or alkaline earth metal hydroxide, filtering the undissolved compound salt, and evaporating and drying the filtrate. At this time, it is pharmaceutically appropriate to prepare a sodium, potassium, or calcium salt as a metal salt. A corresponding silver salt is obtained by allowing an alkali metal or alkaline earth metal salt to react with a suitable silver salt (e.g., silver nitrate).

According to one preferred embodiment of the present invention, the sleep disorder may be one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

The pharmaceutical composition of the present invention may be in various oral or parenteral dosage forms. When formulating the composition, one or more buffering agents (e.g., saline or phosphate-buffered saline (PBS)), antidiabetic agents, bacteriostatic agents, chelating agents (e.g., ethylenediaminetetraacetic acid (EDTA) or glutathione), fillers, bulking agents, binders, adjuvants (e.g., aluminum hydroxide), suspending agents, thickening agents, wetting agents, disintegrants or surfactants, diluents or excipients may be used.

Solid preparations for oral administration include tablets, pills, powder, granules, capsules, and the like, and these solid preparations are prepared by mixing one or more compounds with at least one excipient, such as starch (corn starch, wheat starch, rice starch, potato starch, etc.), calcium carbonate, sucrose, lactose, dextrose, sorbitol, mannitol, xylitol, erythritol maltitol, cellulose, methyl cellulose, sodium carboxymethylcellulose and hydroxypropyl methylcellulose or gelatin. For example, tablets or sugar-coated tablets may be obtained by mixing an active ingredient with a solid excipient, grinding the resulting mixture, adding a suitable adjuvant, and processing the same into a granular mixture. In addition, in addition to a simple excipient, lubricants such as magnesium stearate and talc are also used.

Liquid preparations for oral administration include suspensions, oral liquids, emulsions, or syrups. In addition to commonly used simple diluents such as water and liquid paraffin, various excipients such as wetting agents, sweeteners, fragrances, or preservatives may be included. In addition, in some cases, cross-linked polyvinylpyrrolidone, agar, alginic acid, or sodium alginate may be added as a disintegrant, and anti-coagulants, lubricants, wetting agents, fragrances, emulsifiers, and preservatives may further be included.

Preparations for parenteral administration include sterilized aqueous solutions, non-aqueous solutions, suspensions, emulsions, freeze-dried preparations, or suppositories. As non-aqueous solvents and suspensions, propylene glycol, polyethylene glycol, vegetable oil such as olive oil, and injectable esters such as ethyl oleate may be used. As a base for suppositories, Witepsol, Macrogol, Tween 61, cacao oil, laurel oil, glycerol, gelatin or the like may be used.

The pharmaceutical composition of the present invention may be administered orally or parenterally, and when administered parenterally, it may be formulated in the forms of external skin preparations; injections administered intraperitoneally, rectally, intravenously, intramuscularly, subcutaneously, intrauterineally, or intracerebrovascularly; transdermal administration; or nasal inhalants according to methods known in the art.

The above injections must be sterilized and protected from contamination by microorganisms such as bacteria and fungi. For injections, examples of suitable carriers include, but are not limited to, solvents or dispersion media including water, ethanol, polyols (e.g., glycerol, propylene glycol, and liquid polyethylene glycol, etc.), mixtures thereof, and/or vegetable oils. More preferably, as suitable carriers, Hanks' solution, Ringer's solution, PBS containing triethanolamine, or isotonic solutions such as sterile water for injection, 10% ethanol, 40% propylene glycol, and 5% dextrose may be used. In order to protect the injections from microbial contamination, various antibacterial and antifungal agents such as paraben, chlorobutanol, phenol, sorbic acid, and thimerosal may be further included. In addition, in most cases, the injections may further include an isotonic agent such as sugars or sodium chloride.

For transdermal administration, forms such as ointments, creams, lotions, gels, external liquids, paste preparations, liniments, and aerosols are used. In the above, transdermal administration means that an effective amount of an active ingredient contained in a pharmaceutical composition is delivered into the skin by topically administering the pharmaceutical composition to the skin.

For inhalation administration, extracts used according to the present invention may be conveniently delivered in the form of an aerosol spray from a pressurized pack or nebulizer using a suitable propellant, for example, dichlorofluoromethane, trichlorofluoromethane, dichlorotetrafluoroethane, carbon dioxide or other suitable gases. For pressurized aerosols, the dosage unit may be determined by providing a valve that delivers a metered amount. For example, gelatin capsules and cartridges used in inhalers or insufflators may be formulated to contain a powder mixture of a compound and a suitable powder base such as lactose or starch. Formulations for parenteral administration are described in Remington's Pharmaceutical Science (15th Edition, 1975. Mack Publishing Company, Easton, Pennsylvania 18042, Chapter 87: Blaug, Seymour), which is a reference generally known throughout pharmaceutical chemistry.

The pharmaceutical composition of the present invention is administered in a pharmaceutically effective amount. A pharmaceutically effective amount refers to an amount sufficient to treat a disease at a reasonable benefit/risk ratio applicable to medical treatment, and the effective dose level may be determined according to factors including the type and severity of a patient's disease, drug activity, sensitivity to the drug, administration time, administration route and excretion rate, treatment duration, and concurrently used drugs, and other factors well known in the medical field. The composition of the present invention may be administered as an individual therapeutic agent or in combination with another therapeutic agent, may be administered sequentially or simultaneously with conventional therapeutic agents, and may be administered once or multiple times. In other words, the total effective amount of the composition of the present invention may be administered to a patient in a single dose, or by a fractionated treatment protocol in which multiple doses are administered over a long period of time. It is important to administer an amount that may achieve the maximum effect with the minimum amount without side effects by considering all of the above factors, and this may be easily determined by those skilled in the art to which the present invention pertains.

The range of the administration amount of the pharmaceutical composition of the present invention varies according to a patient's weight, age, sex, health conditions, and diet, administration time, administration method, excretion rate, and severity of the disease. The daily dose may be administered once or in several divided doses such that preferably an amount of 0.01 to 200 mg, more preferably 0.1 to 120 mg per kg of body weight per day may be administered based on the compound represented by [Chemical Formula 1], an isomer thereof, and a pharmaceutically acceptable salt thereof in the case of parenteral administration, and preferably an amount of 0.01 to 200 mg, more preferably 0.01 to 20 mg per kg of body weight per day may be administered based on the compound represented by [Chemical Formula 1], an isomer thereof, and a pharmaceutically acceptable salt thereof in the case of oral administration. However, since it may increase or decrease depending on the administration route, severity of disease, sex, weight, age, etc., the dosage does not limit the scope of the present invention in any way.

The composition of the present invention may be used independently or in combination with surgery, radiotherapy, hormone therapy, chemotherapy, and methods using a biological response modifier.

The pharmaceutical composition of the present invention may be provided in the form of external preparations including the compound represented by [Chemical Formula 1], an isomer thereof, and a pharmaceutically acceptable salt thereof as an active ingredient. When the pharmaceutical composition for sleep improvement or sleep treatment of the present invention is used as an external skin preparation, it may further contain adjuvants commonly used in the field of dermatology such as fatty substances, organic solvents, solubilizing agents, thickening and gelling agents, softeners, antidiabetic agents, suspending agents, stabilizers, foaming agents, flavoring agents, surfactants, water, ionic or non-ionic emulsifiers, fillers, sequestering agents, chelating agents, preservatives, vitamins, blocking agents, wetting agents, essential oils, dyes, pigments, hydrophilic active agents, lipophilic active agents or lipid vesicles or any other ingredients commonly used in external skin preparations. In addition, the ingredients may be introduced in amounts commonly used in the field of dermatology.

When the pharmaceutical composition for sleep improvement or sleep treatment of the present invention is provided as an external skin preparation, it may be in the form of an ointment, patch, gel, cream, or spray, but is not limited thereto.

In addition, the present invention may provide a health functional food composition for preventing or relieving a sleep disorder, including a compound represented by [Chemical Formula 1] below, an isomer thereof, or a pharmaceutically acceptable salt thereof:

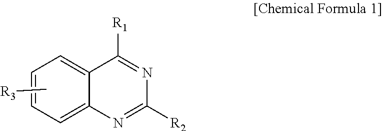

wherein R₁ or R₂ is any one selected from the group consisting of hydrogen, a straight-chain or branched C₁-C₃ alkyl, a halogen,

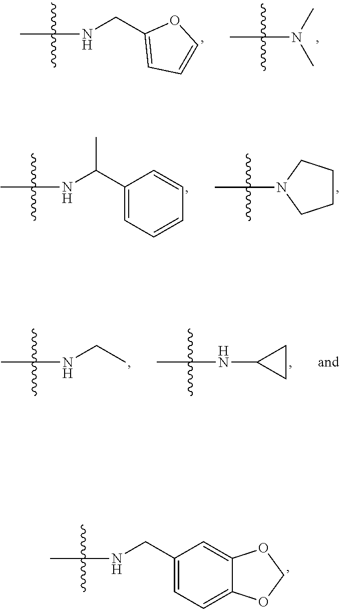

and
R₃ is one or more selected from the group consisting of hydrogen, a straight-chain or branched C₁-C₃ alkyl, a halogen, hydroxy, and methoxy.

Since the compound represented by [Chemical Formula 1], an isomer thereof, and a pharmaceutically acceptable salt thereof are the same as the concepts used in the pharmaceutical composition, the description is replaced with the above description.

According to one preferred embodiment of the present invention, the sleep disorder may be one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

There is no particular limitation on the type of health functional food. Examples include drinks, meat, sausages, bread, biscuits, rice cakes, chocolates, candies, snacks, confectioneries, pizza, ramen, other noodles, gum, dairy products including ice cream, various soups, beverages, alcoholic beverages, vitamin complexes, milk products, and processed milk products, and include all health functional foods in a conventional sense.

The compound represented by [Chemical Formula 1], an isomer thereof, and a pharmaceutically acceptable salt thereof may be added to food as is or used together with other food or food ingredients, and may be appropriately used according to conventional methods. The mixing amount of the active ingredient may be appropriately determined according to the purpose of use (prevention or improvement). Generally, the amount of the compound in health functional food may be 0.1 to 90 parts by weight of the total weight of the food. However, in the case of long-term intake for the purpose of health and hygiene or health control, the amount may be less than the above range, and since there is no problem in terms of safety, the active ingredient may also be used in an amount greater than the above range.

A health functional beverage composition of the present invention has no particular restrictions on other ingredients other than containing the compound of the present invention as an essential ingredient at the indicated proportion, and may contain various flavoring agents or natural carbohydrates as additional ingredients like conventional beverages. Examples of the above-described natural carbohydrates include monosaccharides such as glucose and fructose; disaccharides such as maltose and sucrose; and common sugars such as polysaccharides, for example, dextrin and cyclodextrin, and sugar alcohols such as xylitol, sorbitol, and erythritol. As flavoring agents other than those mentioned above, natural flavoring agents (thaumatin, stevia extract (e.g., rebaudioside A, glycyrrhizin, etc.)) and synthetic flavoring agents (saccharin, aspartame, etc.) may be advantageously used. The proportion of natural carbohydrates is generally about 1 to 20 g, preferably about 5 to 12 g, per 100 g of the composition of the present invention.

In addition to the above, the health functional food composition including a compound represented by [Chemical Formula 1] below, an isomer thereof, or a pharmaceutically acceptable salt thereof according to the present invention may contain various nutrients, vitamins, minerals (electrolytes), flavoring agents such as synthetic flavoring agents and natural flavoring agents, coloring agents and thickening agents (cheese, chocolate, etc.), pectic acid and salts thereof, alginic acid and salts thereof, organic acids, protective colloidal thickening agents, pH adjusters, stabilizers, preservatives, glycerin, alcohols, carbonating agents used in carbonated drinks, and the like. In addition, along with the compound represented by [Chemical Formula 1], an isomer thereof, and a pharmaceutically acceptable salt thereof, the health functional beverage composition may contain fruit flesh for the production of natural fruit juice, fruit juice beverages, and vegetable beverages. These ingredients may be used independently or in combination.

In addition, the present invention may provide a composition for sleep improvement, including a compound represented by [Chemical Formula 1] below, an isomer thereof, or a pharmaceutically acceptable salt thereof:

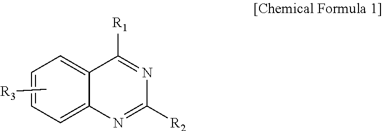

wherein R₁ or R₂ is any one selected from the group consisting of hydrogen, a straight-chain or branched C₁-C₃ alkyl, a halogen,

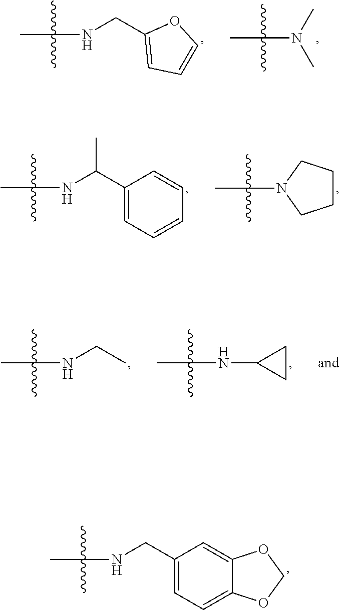

and
R₃ is one or more selected from the group consisting of hydrogen, a straight-chain or branched C₁-C₃ alkyl, a halogen, hydroxy, and methoxy.

Since the compound represented by [Chemical Formula 1], an isomer thereof, and a pharmaceutically acceptable salt thereof are the same as the concepts used in the pharmaceutical composition, the description is replaced with the above description.

The composition of the present invention is preferably a cosmetic composition, fragrance composition, or quasi-drug composition, but is not limited thereto.

When formulated as a cosmetic composition, the content of the compound represented by [Chemical Formula 1], an isomer thereof, and a pharmaceutically acceptable salt thereof is 0.0001% to 10% by weight, preferably 0.01% to 5.0% by weight based on the total weight of the cosmetic composition. In order to achieve the minimum amelioration or prevention effect, the content of the compound represented by [Chemical Formula 1], an isomer thereof, and a pharmaceutically acceptable salt thereof is preferably more than the above minimum value, and considering the reduced feeling of use due to the addition of an excessive amount and the possibility of application to various formulations, the content of the compound represented by [Chemical Formula 1], an isomer thereof, and a pharmaceutically acceptable salt thereof is preferably less than the above maximum value. At this time, it is preferable that the content of the compound represented by [Chemical Formula 1], an isomer thereof, and a pharmaceutically acceptable salt thereof is appropriately adjusted within the above range depending on the content of ingredients contained in the formulation or cosmetic composition.

Ingredients included in the cosmetic composition of the present invention include ingredients commonly used in cosmetic compositions, in addition to the compound represented by [Chemical Formula 1], an isomer thereof, and a pharmaceutically acceptable salt thereof, for example, conventional adjuvants such as antioxidants, stabilizers, solubilizers, vitamins, pigments, and flavors, and carriers.

The cosmetic composition of the present invention may be prepared in any formulation commonly manufactured in the art, for example, it may be formulated into cosmetics such as softening lotion, astringent lotion, nourishing lotion, nourishing cream, massage cream, essence, eye cream, eye essence, cleansing cream, cleansing foam, cleansing water, packs, gel, powder, body lotion, body cream, body oil, and body essence.

When the formulation of the present invention is a paste, cream or gel, animal oil, vegetable oil, wax, paraffin, starch, tragacanth, a cellulose derivative, polyethylene glycol, silicone, bentonite, silica, talc or zinc oxide may be used as a carrier ingredient.

When the formulation of the present invention is a powder or spray, lactose, talc, silica, aluminum hydroxide, calcium silicate, or polyamide powder may be used as a carrier ingredient. In particular, when the formulation is a spray, a propellant such as chlorofluorohydrocarbon, propane/butane, or dimethyl ether may further be included.

When the formulation of the present invention is a solution or emulsion, a solvent, solubilizing agent, or emulsifying agent is used as a carrier ingredient, and examples include water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, or aliphatic esters of sorbitan.

When the formulation of the present invention is a suspension, a liquid diluent such as water, ethanol or propylene glycol, a suspending agent such as ethoxylated isostearyl alcohol, polyoxyethylene sorbitol ester, and polyoxyethylene sorbitan ester, microcrystalline cellulose, aluminum metahydroxide, bentonite, agar, or tragacanth may be used as a carrier ingredient.

When the formulation of the present invention is a surfactant-containing cleansing agent, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, aliphatic alcohol, fatty acid glyceride, fatty acid diethanolamide, vegetable oil, a lanolin derivative, or ethoxylated glycerol fatty acid ester may be used as a carrier ingredient.

The fragrance composition may be included without limitation as long as it is a product that may exhibit a sleep improvement effect due to the compound represented by [Chemical Formula 1], an isomer thereof, and a pharmaceutically acceptable salt thereof and is used for scenting purposes, but it is preferable that one or more products selected from the group consisting of perfumes, scented candles, oils, air fresheners, detergents, and external skin preparations are included. The external skin preparations include all products necessary for bathing, such as soap, face washers, or bath additives, but are not limited thereto.

In addition, the present invention may provide a sleep aid including a compound represented by [Chemical Formula 1] below, an isomer thereof, or a pharmaceutically acceptable salt thereof.

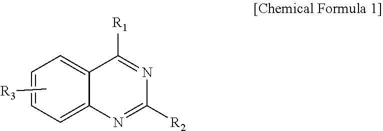

wherein R₁ or R₂ is any one selected from the group consisting of hydrogen, a straight-chain or branched C₁-C₃ alkyl, a halogen,

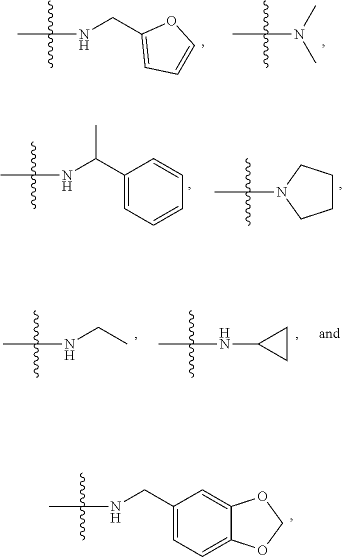

and
R₃ is one or more selected from the group consisting of hydrogen, a straight-chain or branched C₁-C₃ alkyl, a halogen, hydroxy, and methoxy.

Since the compound represented by [Chemical Formula 1], an isomer thereof, and a pharmaceutically acceptable salt thereof are the same as the concepts used in the pharmaceutical composition, the description is replaced with the above description.

The sleep aid refers to one that, unlike sleeping pills, may be purchased without a prescription from a specialist and has a sleep-inducing or sedative effect.

In addition, the prevent invention may provide a compound represented by [Chemical Formula 1] below, an isomer thereof, or a pharmaceutically acceptable salt thereof:

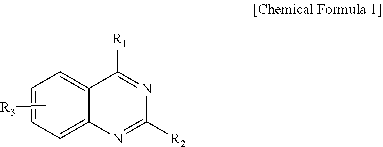

wherein R₁ is any one selected from the group consisting of

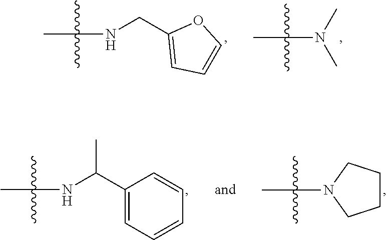

R² is any one selected from the group consisting

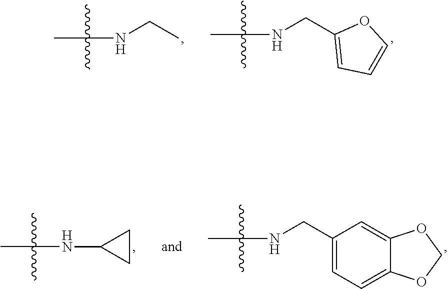

and R₃ is one or more selected from the group consisting of hydrogen, hydroxy, and methoxy.

Since the compound represented by [Chemical Formula 1], an isomer thereof, and a pharmaceutically acceptable salt thereof are the same as the concepts used in the pharmaceutical composition, the description is replaced with the above description.

In addition, the present invention may provide a method of treating a sleep disorder, including administering a compound represented by [Chemical Formula 1] below, an isomer thereof, or a pharmaceutically acceptable salt thereof to a patient with a sleep disorder:

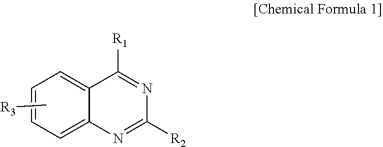

wherein R₁ or R₂ is any one selected from the group consisting of hydrogen, a straight-chain or branched C₁-C₃ alkyl, a halogen,

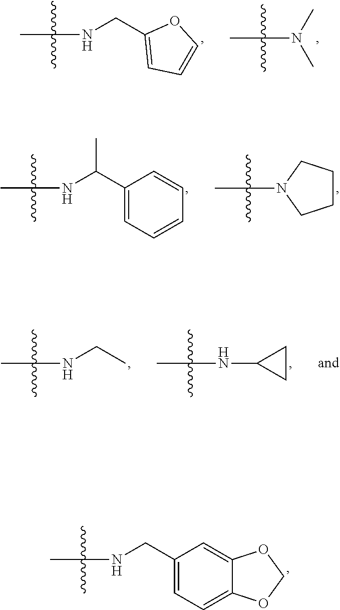

and, and
R₃ is one or more selected from the group consisting of hydrogen, a straight-chain or branched C₁-C₃ alkyl, a halogen, hydroxy, and methoxy.

Since the compound represented by [Chemical Formula 1], an isomer thereof, and a pharmaceutically acceptable salt thereof are the same as the concepts used in the pharmaceutical composition, the description is replaced with the above description.

According to one preferred embodiment of the present invention, the sleep disorder may be one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

In addition, the present invention may provide a use of a compound represented by [Chemical Formula 1] below, an isomer thereof, or a pharmaceutically acceptable salt thereof for treatment of a sleep disorder:

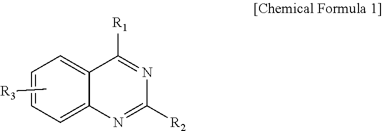

wherein R₁ or R₂ is any one selected from the group consisting of hydrogen, a straight-chain or branched C₁-C₃ alkyl, a halogen,

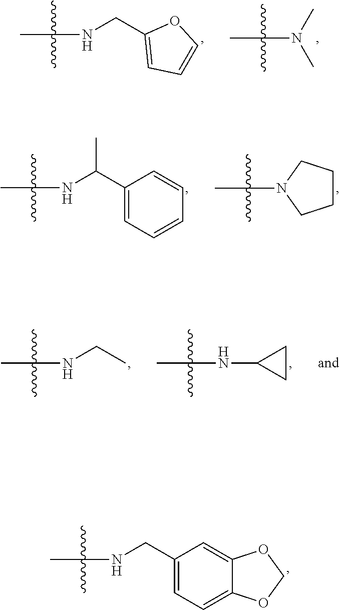

and
R₃ is one or more selected from the group consisting of hydrogen, a straight-chain or branched C₁-C₃ alkyl, a halogen, hydroxy, and methoxy.

Since the compound represented by [Chemical Formula 1], an isomer thereof, and a pharmaceutically acceptable salt thereof are the same as the concepts used in the pharmaceutical composition, the description is replaced with the above description.

According to one preferred embodiment of the present invention, the sleep disorder may be one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

### Advantageous Effects

The novel compound of the present invention can shorten sleep latency and increase total sleep duration, so it can be effectively used in a pharmaceutical composition for preventing or treating a sleep disorder, a health functional food composition for preventing or relieving a sleep disorder, a composition for sleep improvement, a sleep aid or a method of treating a sleep disorder.

### DESCRIPTION OF DRAWINGS

FIG. 1 shows a reaction scheme for synthesizing novel compounds of the present invention.

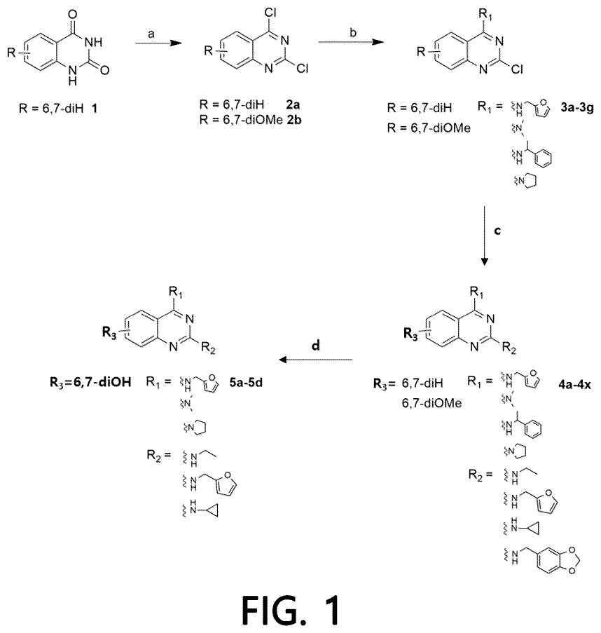

FIG. 2A shows the novel compounds represented by [Chemical Formula 2] to [Chemical Formula 8] of the present invention.

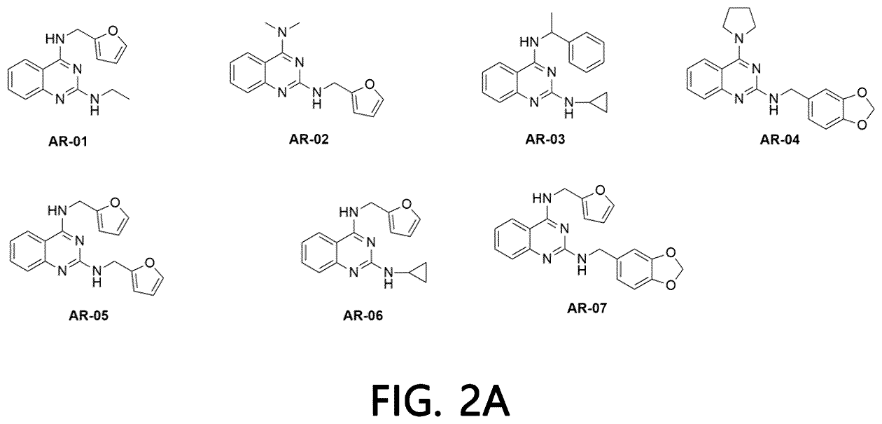

FIG. 2B shows the novel compounds represented by [Chemical Formula 9] to [Chemical Formula 15] of the present invention.

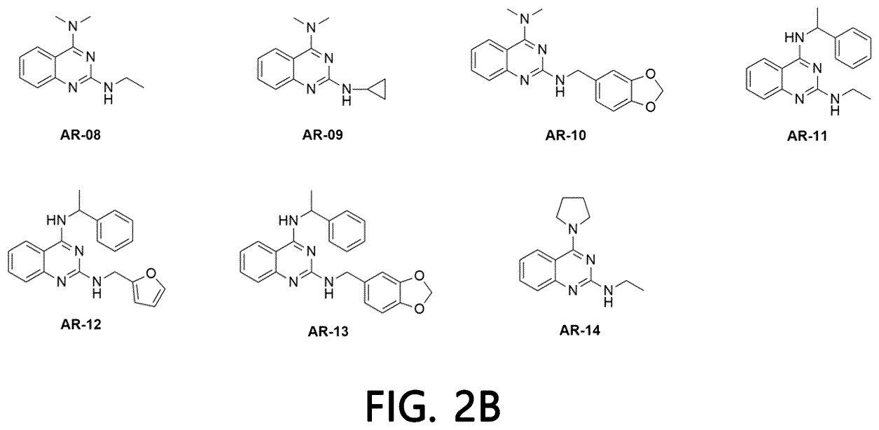

FIG. 2C shows the novel compounds represented by [Chemical Formula 16] to [Chemical Formula 22] of the present invention.

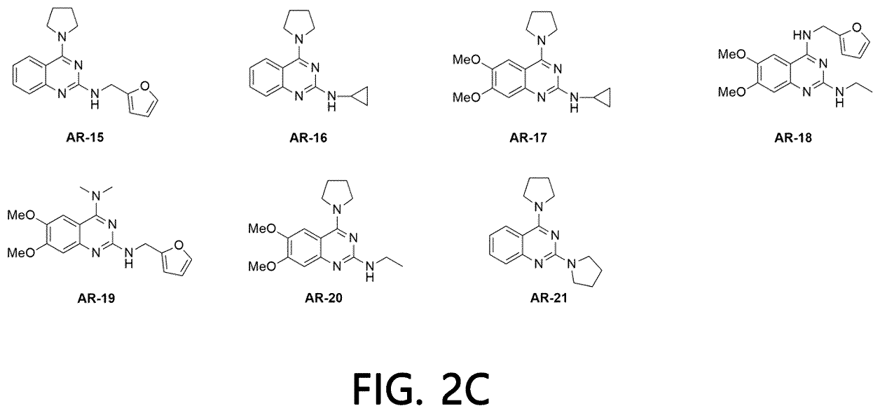

FIG. 2D shows the novel compounds represented by [Chemical Formula 23] to [Chemical Formula 29] of the present invention.

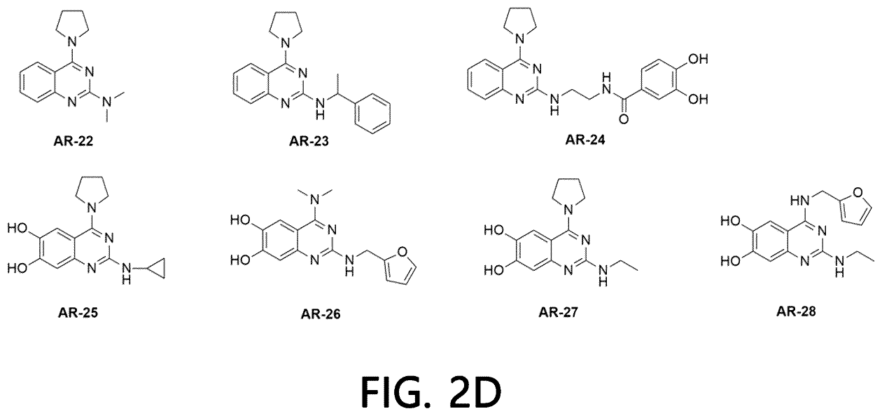

FIG. 3 shows the sleep latency reducing effect of the novel compounds of the present invention.

FIG. 4 shows the total sleep duration increasing effect of the novel compounds of the present invention.

FIG. 5 shows the dose-response experimental results for the compounds that exhibited agonistic activity on an adenosine A1 receptor among the novel compounds of the present invention.

### MODES OF THE INVENTION

### Example 1

### Synthesis of 28 novel compounds

### <1-1> Synthesis of 2,4-dichloroquinazoline (2a)

A solution of benzoyleneurea (2 g, 12.3 mmol) and N,N-dimethylaniline (2.23 g, 12.3 mmol) in phosphorus oxychloride (10 mL) was stirred at a reflux temperature. After 18 hours, the solution was cooled and slowly added to ice cubes and water (90 mL). The formed precipitate was filtered and dissolved in ethyl acetate and chloroform. The organic layer was dried over Na₂SO₄ and concentrated in vacuo. The residue was recrystallized with ethyl acetate and hexane (500 mg, 22.4%). ¹H-NMR (600 MHz, CDCl₃), δ 8.28 (d, J=8.3 Hz, 1H), 8.01 (t, J=2.1 Hz, 2H), 7.75-7.78 (m, 1H).

### <1-2> Synthesis of 2-chloro-N-(furan-2-ylmethyl)quinazolin-4-amine (3a)

Sodium acetate (1.98 g, 24.1 mmol) was added to solution of 2,4-dichloroquinazoline (2 g, 10.0 mmol) and furfurylamine (2.34 g, 24.1 mmol) in THF/H₂O (3/1, 80 mL). The resulting solution was stirred at room temperature for 40 hours. The reaction mixture was diluted with ethyl acetate, and the organic layer was washed with brine and dried over Na₂SO₄. The crude solid was recrystallized with ethyl acetate and hexane (2.5 g, 96.2%). ¹H-NMR (600 MHz, CDCl₃), δ 7.70-7.77 (m, 3H), 7.45 (td, J=7.6, 1.4 Hz, 1H), 7.39 (d, J=1.4 Hz, 1H), 6.35-6.39 (m, 2H), 6.26 (s, 1H), 4.86 (d, J=5.5 Hz, 2H).

### <1-3> Synthesis of 2-chloro-N,N-dimethylquinazolin-4-amine (3b)

Sodium acetate (3.96 g, 48.2 mmol) was added to a solution of 2,4-dichloroquinazoline (2 g, 10.0 mmol) and dimethylamine hydrochloride (1.97 g, 24.1 mmol) in THF/H₂O (3/1, 80 mL). The resulting solution was stirred at room temperature for 40 hours. The reaction mixture was diluted with ethyl acetate, and the organic layer was washed with brine and dried over Na₂SO₄. The crude solid was recrystallized with ethyl acetate and hexane (1.5 g, 71.8%). ¹H-NMR (600 MHz, CDCl₃) δ 8.01 (d, J=8.3 Hz, 1H), 7.67-7.77 (m, 2H), 7.37-7.40 (m, 1H), 3.41 (s, 6H).

### <1-4> Synthesis of 2-chloro-N-(1-phenylethyl)quinazolin-4-amine (3c)

Sodium acetate (1.98 g, 24.1 mmol) was added to a solution of 2,4-dichloroquinazoline (2 g, 10.0 mmol) and DL-phenylmethylamine (3.11 mL, 24.1 mmol) in THF/H₂O (3/1, 80 mL). The resulting solution was stirred at room temperature for 40 hours. The reaction mixture was diluted with ethyl acetate, and the organic layer was washed with brine and dried over Na₂SO₄. The crude solid was recrystallized with ethyl acetate and hexane (1.70 mg, 59.6%). ¹H-NMR (600 MHz, CDCl₃) δ 7.64-7.74 (m, 3H), 7.24-7.42 (m, 6H), 6.31 (s, 1H), 5.61-5.66 (m, 1H) 1.67 (d, J=6.9 Hz, 3H).

### <1-5> Synthesis of 2-chloro-4-(pyrrolidin-1-yl)quinazoline (3d)

Sodium acetate (1.98 g, 24.1 mmol) was added to a solution of 2,4-dichloroquinazoline (2 g, 10.0 mmol) and pyrrolidine (1.98 mL, 24.1 mmol) in THF/H₂O (3/1, 80 mL). The resulting solution was stirred at room temperature for 40 hours. The reaction mixture was diluted with ethyl acetate, and the organic layer was washed with brine, dried over Na₂SO₄, and evaporated in vacuo. The crude solid was recrystallized with ethyl acetate and hexane (2.0 g, 85.1%). ¹H-NMR (600 MHz, CDCl₃) δ 8.11 (d, J=9.0 Hz, 1H), 7.65-7.73 (m, 2H), 7.34-7.37 (m, 1H), 3.93 (s, 4H), 2.04-2.07 (m, 4H).

### <1-6> Synthesis of 2-chloro-6,7-dimethoxy-4-(pyrrolidin-1-yl)quinazoline (3e)

Sodium acetate (1.89 g, 23.2 mmol) was added to a solution of 2,4-dichloro-6,7-dimethoxyquinazoline (2.5 g, 9.65 mmol) and pyrrolidine (1.90 mL, 23.2 mmol) in THF/H₂O (3/1, 80 mL). The resulting solution was stirred at room temperature for 40 hours. The reaction mixture was diluted with ethyl acetate, and the organic layer was washed with brine, dried over Na₂SO₄, and evaporated in vacuo. The crude solid was recrystallized with ethyl acetate and hexane (2.4 g, 84.5%). ¹H-NMR (600 MHz, CDCl₃+CD₃OD) δ 7.47 (s, 1H), 7.04 (s, 1H), 3.94-4.00 (m, 10H), 2.07 (t, J=3.1 Hz, 4H).

### <1-7> Synthesis of 2-chloro-N-(furan-2-ylmethyl)-6,7-dimethoxyquinazolin-4-amine (3f)

Sodium acetate (1.89 g, 23.2 mmol) was added to a solution of 2,4-dichloro-6,7-dimethoxyquinazoline (2.5 g, 9.65 mmol) and furfurylamine (2.04 mL, 23.2 mmol) in THF/H₂O (3/1, 80 mL). The resulting solution was stirred at room temperature for 40 hours. The reaction mixture was diluted with ethyl acetate, and the organic layer was washed with brine, dried over Na₂SO₄, and evaporated in vacuo. The crude solid was recrystallized with ethyl acetate and hexane (2.8 g, 90.6%). ¹H-NMR (600 MHz, CDCl₃) δ 7.33 (s, 1H), 7.09 (s, 1H), 6.98 (s, 1H), 6.34 (d, J=18.6 Hz, 3H), 4.83 (s, 2H), 3.91 (d, J=13.8 Hz, 6H).

### <1-8> Synthesis of 2-chloro-6,7-dimethoxy-N,N-dimethylquinazolin-4-amine (3g)

Sodium acetate (1.89 g, 23.2 mmol) was added to a solution of 2,4-dichloro-6,7-dimethoxyquinazoline (2.5 g, 9.65 mmol) and dimethylamine hydrogen chloride (1.89 g, 23.2 mmol) in THF/H₂O (3/1, 80 mL). The resulting solution was stirred at room temperature for 40 hours. The reaction mixture was diluted with ethyl acetate, and the organic layer was washed with brine, dried over Na₂SO₄, and evaporated in vacuo. The crude solid was recrystallized with ethyl acetate and hexane (2.8 g, 90.6%). ¹H-NMR (600 MHz, CDCl₃) δ 7.24 (s, 1H), 7.14 (s, 1H), 3.98 (d, J=9.0 Hz, 6H), 3.35 (s, 6H).

### <1-9> Synthesis of N²-ethyl-N⁴-(furan-2-ylmethyl)quinazoline-2,4-diamine (4a, AR-01, Chemical Formula 2)

Ethylamine in methanol (4 mL) was added to a solution of 2-chloro-N-(furan-2-ylmethyl)quinazolin-4-amine (400 mg, 2.49 mmol) in methanol in a sealed tube. The resulting solution was heated to 150° C. When the reaction was completed as observed by thin layer chromatography (TLC), the solution was evaporated in vacuo. The residue was purified by column chromatography to obtain the title compound. ¹H-NMR (600 MHz, CD₃OD) δ 7.61 (d, J=8.3 Hz, 1H), 7.26 (dd, J=8.3, 6.9 Hz, 1H), 7.18 (s, 1H), 7.12 (d, J=7.6 Hz, 1H), 6.82 (dd, J=8.1, 7.1 Hz, 1H), 6.05-6.10 (m, 2H), 4.54 (s, 2H), 3.24 (q, J=7.1 Hz, 2H), 0.98 (t, J=7.2 Hz, 4H).

### <1-10> Synthesis of N²-(furan-2-ylmethyl)-N⁴,N-dimethylquinazoline-2,4-diamine, 4b, AR-02, Chemical Formula 3)

Furfurylamine (380 mg, 3.91 mmol) was added to a solution of 2-chloro-N,N-dimethylquinazolin-4-amine (360 mg, 1.73 mmol) in ethanol in a sealed tube. The solution was heated to 150° C. for 4 hours. The mixture was concentrated in vacuo. The formed precipitate was filtered and washed with ethyl acetate. ¹H-NMR (600 MHz, CD₃OD) δ 8.18 (d, J=7.6 Hz, 1H), 7.75 (td, J=7.6, 1.4 Hz, 1H), 7.37-7.45 (m, 3H), 6.36-6.38 (m 2H), 4.71 (s, 2H), 3.55 (s, 6H).

### <1-11> Synthesis of N²-cyclopropyl-N⁴-(1-phenylethyl)guinazoline-2,4-diamine, 4c, AR-03, Chemical Formula 4)

Cyclopropylamine (44.0 L, 0.634 mmol) was added to a solution of 2-chloro-N-(1-phenylethyl)quinazolin-4-amine (60 mg, 0.211 mmol) in ethanol in a sealed tube. The resulting solution was heated to 150° C. When the reaction was completed as observed by TLC, the solution was evaporated in vacuo. The residue was purified by column chromatography to obtain the title compound. ¹H-NMR (600 MHz, CD₃OD) δ 8.13 (d, J=7.6 Hz, 1H), 7.63 (t, J=8.3 Hz, 1H), 7.45 (dd, J=22.4, 7.9 Hz, 3H), 0.3220-7 (m, 4H), 5.63 (q, J=7.1 Hz, 1H), 2.72-2.76 (m, 1H), 1.65 (d, J=6.9 Hz, 3H), 0.77-0.84 (m, 2H), 0.53-0.61 (m, 2H).

### <1-12> Synthesis of N-(benzo[d][1,3]dioxol-5-ylmethyl)-4-(pyrrolidin-1-yl)quinazolin-2-amine (4d, AR-04, Chemical Formula 5)

Piperonylamine (146 mg, 0.963 mmol) was added to a solution of 2-chloro-4-(pyrrolidin-1-yl)quinazoline (150 mg, 0.642 mmol) in ethanol in a sealed tube. The resulting solution was heated to 150° C. After 18 hours, the solution was evaporated in vacuo. The formed precipitate was filtered. ¹H-NMR (600 MHz, DMSO-d₆) δ 8.03 (d, J=8.3 Hz, 1H), 7.47 (t, J=7.6 Hz, 1H), 7.27 (d, J=8.3 Hz, 1H), 6.94-7.02 (m, 2H), 6.83 (t, J=9.3 Hz, 2H), 5.96 (s, 2H), 4.45 (d, J=6.9 Hz, 2H), 3.80 (t, J=6.2 Hz, 4H), 1.94 (t, J=6.5 Hz, 4H).

### <1-13> Synthesis of N² N⁴-bis(furan-2-ylmethyl)quinazoline-2,4-diamine (4e, AR-05, Chemical Formula 6)

Furfurylamine (306 L, 3.47 mmol) was added a solution of 2-chloro-N-(furan-2-ylmethyl)quinazolin-4-amine (300 mg, 1.16 mmol) in ethanol (14 mL) in a sealed tube. The resulting solution was heated to 150° C. When the reaction was completed as observed by TLC, the solution was evaporated in vacuo. The crude product was purified by recrystallization with methanol. ¹H-NMR (600 MHz, CDCl₃+CD₃OD) δ 8.02 (d, J=8.3 Hz, 1H), 7.65 (d, J=7.6 Hz, 1H), 7.31-7.49 (m, 5H), 6.27-6.33 (m, 4H), 4.83 (s, 2H), 4.71 (s, 2H), 4.48 (s, 12H).

### <1-14> Synthesis of N²-cyclopropyl-N⁴-(furan-2-ylmethyl)quinazoline-2,4-diamine (4f, AR-06, Chemical Formula 7)

Cyclopropylamine (66.7 L, 0.963 mmol) was added to a solution of 2-chloro-N-(furan-2-ylmethyl)quinazolin-4-amine (250 mg, 0.963 mmol) in ethanol (14 mL) in a sealed tube. The solution was heated to 150° C. When the reaction was completed as observed by TLC, the solution was evaporated in vacuo. The crude product was purified by recrystallization with ethyl acetate. ¹H-NMR (600 MHz, CDCl₃+CD₃OD) δ 8.07 (d, J=8.3 Hz, 1H), 7.71 (s, 1H), 7.36 (s, 2H), 6.32-6.35 (m, 2H), 4.83 (s, 1H), 3.06-2.56 (1H), 0.73 (s, 4H).

### <1-15> Synthesis of N²-(benzo[d][1,3]dioxol-5-ylmethyl)-N⁴-furan-2-ylmethyl)quinazoline-2,4-diamine (4g, AR-07, Chemical Formula 8)

Piperonylamine (119 L, 0.963 mmol) was added to a solution of 2-chloro-N-(furan-2-ylmethyl)quinazolin-4-amine (250 mg, 0.963 mmol) in ethanol (14 mL) in a sealed tube. The resulting solution was heated to 150° C. When the reaction was completed as observed by TLC, the solution was evaporated in vacuo. The crude product was purified by recrystallization with ethyl acetate. ¹H-NMR (600 MHz, CDCl₃+CD₃OD) δ 8.04 (d, J=8.3 Hz, 1H), 7.68-7.71 (m, 1H), 7.33-7.36 (m, 3H), 6.72-6.84 (m, 3H) 6.23-6.31 (m, 2H), 5.91 (s, 2H), 4.81 (s, 2H), 4.63 (s, 2H).

### <1-16> Synthesis of N²-ethyl-N⁴,N⁴-dimethylquinazoline-2,4-diamine (4h, AR-08, Chemical Formula 9)

Ethylamine in THE (603 μL, 1.20 mmol) was added to a solution of 2-chloro-N,N-dimethylquinazolin-4-amine (250 mg, 1.20 mmol) in ethanol (12 mL) in a sealed tube. The resulting solution was heated to 150° C. When the reaction was completed as observed by TLC, the solution was evaporated in vacuo. The crude product was purified by recrystallization with ethyl acetate and methylene chloride. ¹H-NMR (600 MHz, CDCl₃) δ 8.23 (s, 1H), 7.90 (d, J=7.6 Hz, 1H), 7.58-7.64 (m, 2H), 7.23-7.26 (m, 1H), 3.45 (m, 1H), 3.49-3. (m, 8H), 1.29 (t, J=7.2 Hz, 3H).

### <1-17> Synthesis of N²-cyclopropyl-N⁴ N⁴-dimethylquinazoline-2,4-diamine (4i, AR-09, Chemical Formula 10)

Cyclopropylamine (83.4 L, 1.20 mmol) was added to a solution of 2-chloro-N,N-dimethylquinazolin-4-amine (250 mg, 1.20 mmol) in ethanol (12 mL) in a sealed tube. The resulting solution was heated to 150° C. When the reaction was completed as observed by TLC, the solution was evaporated in vacuo. The crude product was purified by recrystallization with ethyl acetate and methanol (10/1). ¹H-NMR (600 MHz, CD₃OD) δ 8.20 (d, J=8.3 Hz, 1H), 7.69-7.80 (m, 1H), 7.44 (t, J=8.3 Hz, 1H), 3.513 (s, 5H), 2.8 (s, 1H), 0.98 (d, J=4.1 Hz, 2H), 0.71-0.76 (m, 2H).

### <1-18> Synthesis of (N²-(benzo[d][1,3]dioxol-5-ylmethyl)-N⁴ N⁴-dimethylquinazoline-2,4-diamine (4j, AR-10, Chemical Formula 11)

Piperonylamine (179 L, 1.44 mmol) was added to a solution of 2-chloro-N,N-dimethylquinazolin-4-amine (300 mg, 1.44 mmol) in ethanol (12 mL) in a sealed tube. The resulting solution was heated to 150° C. When the reaction was completed as observed by TLC, the solution was evaporated in vacuo. The crude product was stirred in methanol, filtered, and washed with methanol. ¹H-NMR (600 MHz, CDCl₃+CD₃OD) δ 7.92 (t, J=7.6 Hz, 1H), 7.55-7.66 (m, 2H), 7.25-7.36 (m, 1H), 6.69-6.82 (m, 3H), 5.88 (s, 2H), 4.55 (s, 2H), 3.47 (s, 6H).

### <1-19> Synthesis of N²-ethyl-N⁴-(1-phenylethyl)quinazoline-2,4-diamine (4k, AR-11, Chemical Formula 12)

Ethylamine in THE (441 L, 0.881 mmol) was added to a solution of 2-chloro-N-(1-phenylethyl)quinazolin-4-amine (250 mg, 0.881 mmol) in ethanol (12 mL) in a sealed tube. The resulting solution was heated to 150° C. When the reaction was completed as observed by TLC, the solution was evaporated in vacuo.

The crude product was purified by recrystallization with methanol and ethyl acetate. ¹H-NMR (600 MHz, CDCl₃+CD₃OD) δ 8.25 (d, J=8.3 Hz, 1H), 7.69 (t, J=7.9 Hz, 1H), 7.22-7.39 (m, 7H), 5.53 (s, 1H), 3.40-3.49 (m, 2H), 1.69 (d, J=6.9 Hz, 3H), 1.14-1.17 (m, 3H).

### <1-20> Synthesis of N²-(furan-2-ylmethyl)-N⁴-(1-phenylethyl)quinazoline-2,4-diamine (4I, AR-12, Chemical Formula 13)

Furfurylamine (77.8 L, 0.881 mmol) was added to a solution of 2-chloro-N-(1-phenylethyl)quinazolin-4-amine (25 0 mg, 0.881 mmol) in ethanol (12 mL) in a sealed tube. The resulting solution was heated to 150° C. When the reaction was completed as observed by TLC, the solution was evaporated in vacuo. The crude product was stirred in methanol, filtered, and washed with methanol. 1H-NMR (600 MHz, CDCl₃+CD₃OD) δ 8.26-8.28 (m, 1H), 7.70-7.72 (m, 1H), 7.23-7.40 (m, 8H), 6.29 (q, J=1.6 Hz, 1H) 6.15 (s, 1H), 5.60 (q, J=7.1 Hz, 1H), 1.69 (d, J=7.6 Hz, 3H).

### <1-21> Synthesis of N²-(benzo[d][1,3]dioxol-5-ylmethyl)-N⁴-(1-phenylethyl)quinazoline-2,4-diamine (4m, AR-13, Chemical Formula 14)

Piperonylamine (109 L, 0.881 mmol) was added to a solution of 2-chloro-N-(1-phenylethyl)quinazolin-4-amine (250 mg, 0.881 mmol) in ethanol (12 mL) in a sealed tube. The resulting solution was heated to 150° C. When the reaction was completed as observed by TLC, the solution was evaporated in vacuo. The crude product was stirred in methanol, filtered, and washed with methanol. ¹H-NMR (600 MHz, CDCl₃+CD₃OD) δ 8.27 (d, J=8.3 Hz, 1H), 7.71 (t, J=7.9 Hz, 1H), 7.21-7.39 (m, 7H), 6.67 (s, 3H), 5.89-5.91 (m, 2H), 5.53 (d, J=6.9 Hz, 1H), 4.59 (d, J=14.5 Hz, 2H), 4.44 (d, J=14.5 Hz, 1H), 1.67 (d, J=6.9 Hz, 3H).

### <1-22> Synthesis of N-ethyl-4-(pyrrolidin-1-yl)quinazolin-2-amine (4n, AR-14, Chemical Formula 15)

Ethylamine in THE (578 L, 1.16 mmol) was added to a solution of 2-chloro-4-(pyrrolidin-1-yl)quinazoline (270 mg, 1.16 mmol) in ethanol (14 mL) in a sealed tube. The resulting solution was heated to 150° C. When the reaction was completed as observed by TLC, the solution was evaporated in vacuo. The crude product was purified by recrystallization with hexane and ethyl acetate. ¹H-NMR (600 MHz, CDCl₃) δ 8.11 (s, 1H), 7.98 (d, J=8.3 Hz, 1H), 7.54-7.60 (m, 2H), 7.20-7.23 (m, 1H), 3.99 (m, 4H), 3.49 (dt, J=14.0, 6.5 Hz, 2H), 2.08 (m, 4H), 1.25 (q, J=6.9 Hz, 3H).

### <1-23> Synthesis of N-(furan-2-ylmethyl)-4-(pyrrolidin-1-yl)quinazolin-2-amine (4o, AR-15, Chemical Formula 16)

Furfurylamine (103 L, 1.16 mmol) was added to a solution of 2-chloro-4-(pyrrolidin-1-yl)quinazoline (270 mg, 1.16 mmol) in ethanol (14 mL) in a sealed tube. The solution was heated to 150° C. When the reaction was completed as observed by TLC, the solution was evaporated in vacuo. The crude product was purified by recrystallization with hexane and ethyl acetate. The crude product was stirred in methanol, filtered, and washed with methanol. ¹H-NMR (400 MHz, CDCl₃+CD₃OD) δ 8.20-8.08 (d, J=8.4 Hz, 1H), 7.74-7.64 (t, J=7.6 Hz, 1H), 7.52-7.44 (d, J=8.4 Hz, 1H), 7.40-7.30 (m, 2H), 6.36-6.22 (m, 2H), 4.67 (s, 2H), 4.30-3.80 (m, 4H), 2.20-1.95 (s, 4H).

### <1-24> Synthesis of N-cyclopropyl-4-(pyrrolidin-1-yl)quinazolin-2-amine (4p, AR-16, Chemical Formula 17)

Cyclopropylamine (80 L, 1.16 mmol) was added to a solution of 2-chloro-4-(pyrrolidin-1-yl)quinazoline (270 mg, 1.16 mmol) in ethanol (14 mL) in a sealed tube. The resulting solution was heated to 150° C. When the reaction was completed as observed by TLC, the solution was evaporated in vacuo. ¹H-NMR (600 MHz, CDCl₃) δ 8.01 (d, J=8.3 Hz, 1H), 7.58-7.65 (m, 2H), 7.23-7.25 (m, 1H), 4.04 (s, 4H), 2.82 (s, 1H), 2.11 (d, J=22.7 Hz, 4H), 0.71-0.82 (m, 4H).

### <1-25> Synthesis of N-cyclopropyl-6,7-dimethoxy-4-(pyrrolidin-1-yl)quinazolin-2-amine (4u, AR-17, Chemical Formula 18)

Cyclopropyl (59 L, 0.851 mmol) was added to a solution of 2-chloro-6,7-dimethoxy-4-(pyrrolidin-1-yl)quinazoline (250 mg, 0.851 mmol) in ethanol (14 mL) in a sealed tube. The resulting solution was heated to 150° C. When the reaction was completed as observed by TLC, the solution was evaporated in vacuo. ¹H-NMR (600 MHz, CDCl₃) δ 8.00 (s, 1H), 7.42 (s, 1H), 6.91 (s, 1H), 3.90-4.10 (m, 10H), 2.80 (s, 1H), 2.02-2.18 (m, 4H), 0.68-0.88 (m, 4H).

### <1-26> Synthesis of N²-ethyl-N⁴-(furan-2-ylmethyl)-6,7-dimethoxyguinazoline-2,4-diamine (4v, AR-18, Chemical Formula 19)

Ethylamine (2 M solution, 2.35 mL, 2.35 mmol) was added to a solution of 2-chloro-N-(furan-2-ylmethyl)-6,7-dimethoxy-4-amine (750 mg, 2.35 mmol) in ethanol (14 mL) in a sealed tube. The solution was heated to 150° C. When the reaction was completed as observed by TLC, the solution was evaporated in vacuo. ¹H-NMR (600 MHz, CDCl₃+CD₃OD) δ 7.56 (s, 1H), 7.41 (s, 1H), 6.91 (s, 1H), 6.34-6.36 (m, 2H), 3.97 (s, 3H), 3.92 (s, 3H), 3.54 (d, J=6.9 Hz, 2H), 1.29 (t, J=7.2 Hz, 3H).

### <1-27> Synthesis of N²-(furan-2-ylmethyl)-6,7-dimethoxy-N⁴N⁴_-dimethylquinazoline-2,4-diamine (4w, AR-19, Chemical Formula 20)

Furfurylamine (247 L, 2.80 mmol) was added to a solution of 2-chloro-6,7-dimethoxy-N,N-dimethylquinazolin-4-amine (750 mg, 2.80 mmol) in ethanol (14 mL) in a sealed tube. The resulting solution was heated to 150° C. When the reaction was completed as observed by TLC, the solution was evaporated in vacuo. ¹H-NMR (600 MHz, CDCl₃) δ 8.24 (s, 1H), 7.33 (s, 1H), 7.25 (s, 1H), 6.96 (s, 1H), 6.31 (t, J=2.4 Hz, 2H), 4.66 (d, J=6.2 Hz, 2H), 3.95 (s, 6H), 3.49 (s, 6H).

### <1-28> Synthesis of N-ethyl-6,7-dimethoxy-4-(pyrrolidin-1-yl)quinazolin-2-amine (4x, AR-20, Chemical Formula 21)

Ethylamine (2 M solution, 1.28 mL, 2.55 mmol) was added to a solution of 2-chloro-6,7-dimethoxy-4-(pyrrolidin-1-yl)quinazoline (750 mg, 2.55 mmol) in ethanol (14 mL) in a sealed tube. The resulting solution was heated to 150° C. When the reaction was completed as observed by TLC, the solution was evaporated in vacuo. ¹H-NMR (600 MHz, CDCl₃) δ 7.83 (s, 1H), 7.40 (s, 1H), 6.91 (s, 1H), 3.92-4.06 (m, 10H), 3.46-3.51 (m, 2H), 2.06-2.11 (m, 4H), 1.27 (t, J=7.2 Hz, 3H).

### <1-29> Synthesis of 2,4-di(pyrrolidin-1-yl)quinazoline (4q, AR-21, Chemical Formula 22)

Pyrrolidine (264 L, 3.21 mmol) was added to a solution of 2-chloro-4-(pyrrolidin-1-yl)quinazoline (750 mg, 3.21 mmol) in ethanol (14 mL) in a sealed tube. The resulting solution was heated to 150° C. When the reaction was completed as observed by TLC, the solution was evaporated in vacuo. ¹H-NMR (600 MHz, CD₃OD) δ 8.20 (d, J=8.3 Hz, 1H), 7.58-7.69 (m, 2H), 7.29-7.32 (m, 1H), 3.99 (t, J=6.2 Hz, 4H), 3.67 (s, 4H), 2.07-2.09 (m, 8H).

### <1-30> Synthesis of N,N-dimethyl-4-(pyrrolidin-1-yl)quinazolin-2-amine (4r, AR-22, Chemical Formula 23)

Dimethylamine (2 M ethanol solution, 3.21 mL, 3.21 mmol) was added to a solution of 2-chloro-4-(pyrrolidin-1-yl)quinazoline (750 mg, 3.21 mmol) in ethanol (14 mL) in a sealed tube. The resulting solution was heated to 150° C. When the reaction was completed as observed by TLC, the solution was evaporated in vacuo. ¹H-NMR (600 MHz, CD₃OD) δ 8.28-8.29 (m, 1H), 7.77 (td, J=7.9, 1.4 Hz, 1H), 7.67 (d, J=7.6 Hz, 1H), 7.42-7.45 (m, 1H), 3.96-4.16 (m, 4H), 3.35 (s, 6H), 2.06 (m, 4H).

### <1-31> Synthesis of N-(1-phenylethyl)-4-(pyrrolidin-1-vl)quinazolin-2-amine (4s, AR-23, Chemical Formula 24)

DL-phenylmethylamine (414 L, 3.21 mmol) was added to a solution of 2-chloro-4-(pyrrolidin-1-yl)quinazoline (750 mg, 3.21 mmol) in ethanol (14 mL) in a sealed tube. The resulting solution was heated to 150° C. When the reaction was completed as observed by TLC, the solution was evaporated in vacuo. ¹H-NMR (600 MHz, CDCl₃) δ 8.86 (d, J=6.9 Hz, 1H), 7.94 (d, J=9.0 Hz, 1H), 7.54-7.62 (m, 2H), 7.20-7.42 (m, 7H), 5.11 (t, J=6.9 Hz, 1H), 4.02 (dd, J=15.5, 6.5 Hz, 2H), 3.81-3.86 (m, 1H), 3.50 (t, J=6.5 Hz, 1H), 1.95-2.13 (m, 4H), 1.70 (s, 1H), 1.63 (d, J=6.9 Hz, 3H).

### <1-32> Synthesis of 3,4-dihydroxy-N-(2-((4-(pyrrolidin-1-yl)quinazolin-2-yl)amino) ethyl)benzamide (4t, AR-24, Chemical Formula 25)

Tert-butyl (2-aminoethyl)carbamate (414 L, 3.21 mmol) was added to a solution of 2-chloro-4-(pyrrolidin-1-yl)quinazoline (500 mg, 2.14 mmol) in ethanol (14 mL) in a sealed tube. The resulting solution was heated to 150° C. When the reaction was completed as observed by TLC, the solution was evaporated in vacuo to obtain tert-butyl (2-((4-(pyrrolidin-1-yl)quinazolin-2-yl)amino)ethyl)carbamate (500 mg, 65.4%). ¹H-NMR (600 MHz, CDCl₃) δ 8.36 (s, 1H), 8.03 (d, J=9.0 Hz, 1H), 7.56-7.65 (m, 2H), 7.27 (t, J=7.6 Hz, 1H), 5.36 (s, 1H), 4.02 (m, 4H), 3.62 (m, 2H), 3.41 (m, 2H), 2.08-2.18 (m, 4H), 1.42 (s, 9H).

Tert-butyl (2-((4-(pyrrolidin-1-yl)quinazolin-2-yl)amino)ethyl)carbamate (300 mg, 0.839 mmol) was added to 1,4-dioxane containing 4 N hydrogen chloride (4 N HCl in dioxane, 20 mL). The reaction was terminated after 18 hours, the solvent was removed by distillation under reduced pressure, and the solid was filtered to obtain 2-((4-(pyrrolidin-1-yl)quinazolin-2-yl)amino)ethanaminium chloride (230 mg, 93.1%). ¹H-NMR (600 MHz, DMSO-d₆) δ 8.26 (d, J=8.3 Hz, 1H), 8.09 (s, 3H), 7.94 (s, 1H), 7.80 (t, J=7.9 Hz, 1H), 7.56 (s, 1H), 7.40 (t, J=7.6 Hz, 1H), 4.13 (s, 2H), 3.88 (s, 2H), 3.67-3.74 (m, 2H), 3.05 (m, 2H), 1.95-2.05 (m, 4H).

2-((4-(Pyrrolidin-1-yl)quinazolin-2-yl)amino)ethanaminium chloride (100 mg, 0.340 mmol), 3,4-bis((2-methoxyethoxy)methoxy)benzoic acid (112 mg, 0.340 mmol), 1H-benzotriazol-1-yloxytris(dimethylamino)phosphonium hexafluorophosphate (BOP) (181 mg, 0.408 mmol), and triethylamine (142 L, 1.02 mmol) were stirred in dimethylformamide (5 mL) for 18 hours. The product was purified through column chromatography to obtain 3,4-bis((2-methoxyethoxy)methoxy)-N-(2-((4-(pyrrolidin-1-yl)quinazolin-2-yl)amino)ethyl)benzamide (50 mg, 25.8%). ¹H-NMR (CD₃OD) δ 8.22 (d, J=7.6 Hz, 1H), 7.76 (t, J=7.2 Hz, 1H), 7.53 (s, 1H), 7.40 (s, 3H), 7.16 (d, J=7.6 Hz, 1H), 5.31 (s, 2H), 5.21 (s, 2H), 4.08 (s, 2H), 3.65-3.88 (m, 10H), 3.53-3.56 (m, 4H), 1.98-2.06 (m, 5H).

3,4-Bis((2-methoxyethoxy)methoxy)-N-(2-((4-(pyrrolidin-1-yl)quinazolin-2-yl)amino)ethyl)benzamide (50 mg, 0.088 mmol) with carbon tetrabromide (3 mg, 0.009 mmol) was refluxed in 2-propanol (10 mL). After the reaction was completed, the solvent was removed to obtain a compound. ¹H-NMR (600 MHz, CD₃OD) δ 8.19 (s, 1H), 7.75 (d, J=7.6 Hz, 1H), 7.39 (s, 2H), 7.15 (d, J=51.0 Hz, 2H), 6.70 (s, 1H), 4.07 (s, 2H), 3.87 (s, 2H), 3.75 (s, 2H), 3.63 (s, 2H), 2.70 (d, J=3.4 Hz, 1H), 1.97-2.06 (m, 4H), 1.30-1.32 (m, 1H).

### <1-33> Synthesis of 2-(cyclopropylamino)-4-(pyrrolidin-1-vl)quinazoline-6,7-diol (5a, AR-25, Chemical Formula 26)

Boron tribromide (1 M solution, 9.54 mL, 9.54 mmol) was slowly added dropwise to a solution of N-cyclopropyl-6,7-dimethoxy-4-(pyrrolidin-1-yl)quinazolin-2-amine (500 mg, 1.59 mmol) in dichloromethane (30 mL) at −15° C.

After the reaction was completed, the reaction mixture was neutralized with a saturated aqueous sodium bicarbonate solution, the resulting solid was obtained through filtration. ¹H-NMR (600 MHz, CD₃OD) δ 7.42 (s, 1H), 7.27 (s, 1H), 3.93-4.06 (m, 4H), 2.89-2.68 (m, 1H), 2.06-2.12 (m, 4H), 0.70-0.80 (m, 4H).

### <1-34> Synthesis of 4-(dimethylamino)-2-((furan-2-ylmethyl)amino)quinazoline-6,7-diol (5b, AR-26, Chemical Formula 27)

Boron tribromide (1 M solution, 9.54 mL, 9.54 mmol) was slowly added dropwise to a solution of N²-(furan-2-ylmethyl)-6,7-dimethoxy-N⁴,N⁴-dimethylquinazoline-2,4-diamine (500 mg, 1.59 mmol) in dichloromethane (30 mL) at −15° C. After the reaction was completed, the reaction mixture was neutralized with a saturated aqueous sodium bicarbonate solution, the resulting solid was obtained through filtration. ¹H-NMR (600 MHz, CD₃OD) δ 7.42 (d, J=10.3 Hz, 2H), 7.02 (s, 1H), 6.33-6.36 (m, 2H), 4.69 (s, 2H), 3.55 (s, 6H).

### <1-35> Synthesis of 2-(ethylamino)-4-(pyrrolidin-1-vl)quinazoline-6,7-diol (5c, AR-27, Chemical Formula 28)

Boron tribromide (1 M solution, 9.92 mL, 9.92 mmol) was slowly added dropwise to a solution of N-ethyl-6,7-dimethoxy-4-(pyrrolidin-1-yl)quinazolin-2-amine (500 mg, 1.65 mmol) in dichloromethane (30 mL) at −15° C. After the reaction was completed, the reaction mixture was neutralized with a saturated aqueous sodium bicarbonate solution, the resulting solid was obtained through filtration. ¹H-NMR (600 MHz, DMSO-d₆) δ 7.06 (s, 1H), 6.46 (s, 1H), 3.86 (s, 4H), 3.37 (q, J=7.1 Hz, 2H), 1.95 (d, J=20.7 Hz, 5H), 1.16 (t, J=7.2 Hz, 3H).

### <1-36> Synthesis of 2-(ethylamino)-4-((furan-2-ylmethyl)amino)quinazoline-6,7-diol (5d, AR-28, Chemical Formula 29)

Boron tribromide (1 M solution, 9.54 mL, 9.54 mmol) was slowly added dropwise to a solution of N-cyclopropyl-6,7-dimethoxy-4-(pyrrolidin-1-yl)quinazolin-2-amine (500 mg, 1.59 mmol) in dichloromethane (30 mL) at −15° C. After the reaction was completed, the reaction mixture was neutralized with a saturated aqueous sodium bicarbonate solution, the resulting solid was obtained through filtration. ¹H-NMR (600 MHz, CD₃OD) δ 7.39 (s, 1H), 7.09 (s, 1H), 6.99 (s, 1H), 6.24-6.32 (m, 2H), 4.74 (d, J=11.0 Hz, 2H), 3.45 (t, J=6.2 Hz, 2H), 1.19-1.22 (m, 3H).

### Example 2

### Sleep Improvement Efficacy of Novel Compounds

The test groups were divided into a control group (physiological saline solution) and groups orally administered the 28 novel compounds at 10 mg/kg, and the main experiment (sleep induction experiment) was conducted 30 minutes after administration. In the sleep induction experiment, 30 minutes after test substance administration, pentobarbital, which is a tranquilizer, was intraperitoneally administered at 45 mg/kg to induce sleep, and the sleep latency and the total sleep duration were measured to compare the control group and the novel compound administration groups.

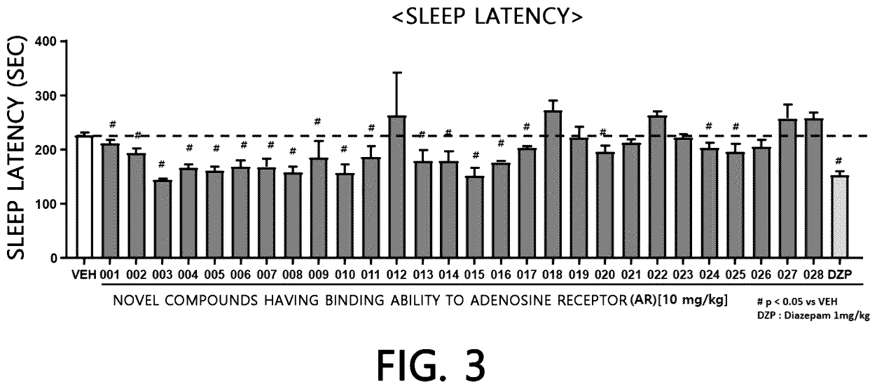

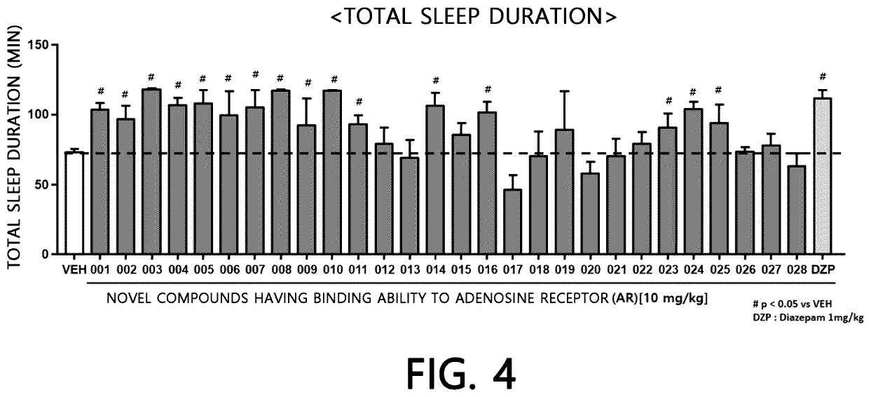

As a result, as shown in FIG. 3 and FIG. 4, it was confirmed that the novel compounds of the present invention exhibited the effect of significantly reducing sleep latency and increasing total sleep duration.

### Example 3

### Mechanism Study on Novel Compounds: Adenosine A1 Receptor Mechanism Study

The effect of adenosine is mediated through at least four specific cell membrane receptors that have been identified to date and classified as receptors A1, A2A, A2B, and A3, belonging to the G protein-coupled receptor family. Through these receptors, adenosine regulates a wide range of physiological functions. To verify the mechanism for the sleep improvement effect, an agonistic effect of the novel compounds on the A1 subtype receptor was confirmed.

The intracellular endoplasmic reticulum has the function of storing calcium ions and releasing them when necessary. It is responsible for managing and maintaining the overall physiological activities of the human body, such as contracting muscles and promoting hormone secretion by releasing calcium ions. This mechanism has been applied as a universal and accurate method to study the activity of G protein-coupled receptors (GPCRs), and the method of measuring calcium ions, which are the final measurement target, has been developed into various methods including fluorescent substances. The present inventors tried to use chemical luminescence through aequorin to evaluate the agonistic efficacy of the compounds subject to development. To this end, a cell line overexpressing an adenosine receptor (adenosine receptor subtype 1, A1), which targets calcium ions released from the intracellular endoplasmic reticulum, was used, and the concentration of calcium ions released instantaneously was measured using a cell function screening system based on the luminescence phenomenon through mitochondrial aequorin.

Cells into which an A1 receptor gene was inserted through transformation were washed with 10 ml of PBS after 48 hours of culture, and then the cells were isolated and centrifuged at 1,000×g for 5 minutes. The precipitated cells were treated with a basic buffer solution (Dulbecco's Modified Eagle's Medium (DMEM)/HAM's F12 without phenol red, with L-glutamate, 15 mM hydroxyethyl piperazine ethane sulfonic acid (HEPES), pH 7.0, 0.1% bovine serum albumin (BSA)), coelenterazine h was added to a concentration of 2×10⁶ cells/ml, and then light was blocked. The cells were allowed to react with coelenterazine h using a rotator at room temperature. After four hours of reaction, the reaction mixture was diluted 10 times with a basic buffer solution to adjust the concentration to 2×10⁵ cells/ml and allowed to stand for 60 minutes under the same conditions as before.

To confirm the agonistic activity of the novel compounds of the present invention, the previously prepared cells were plated in a 96-well black plate (Corning 3915) containing adenosine (10 M-4.57 nM) at 50 l/well (5,000 cells/well), and then the resulting signal value was measured. To confirm the calcium signal changing activity (agonistic activity) of the compounds subject to development, the prepared cells were plated in a 96-well plate containing 50 μl/well of the compound sample at 50 μl/well and allowed to react for 30 minutes. Thereafter, 50 μl/well of adenosine (3 μM) was injected to confirm the reaction. The light emitted due to cell activity was measured and recorded using a Mithras 960 MultiLabel Reader. The measured value (area under the curve (AUC) integrated signal) was expressed in relative light units (RLU). To ensure the accuracy of the experimental data, the experiment was performed at at least two concentrations (10 M and 50 μM) with three samples of the same compound, and EC50 values calculated when necessary were compared and analyzed with the values obtained through at least three experiments.

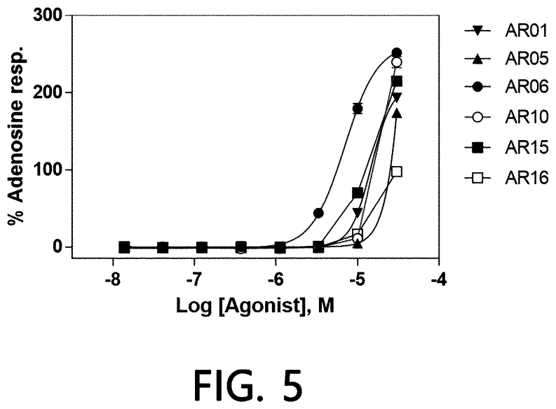

As a result, the results shown in Table 1 below and FIG. 5 were obtained. Each value was obtained by assuming the response of 10 M adenosine as 100% and converting the ratio into percentage. The greater the value, the greater the agonistic effect. The novel compounds of the present invention were confirmed to exhibit an agonistic effect on the A1 receptor at a concentration of 50 PM. In particular, for the AR-06 compound, the EC50 value was measured to be 6.95 μM.

TABLE 1

Concentration

| Compound | 10 μM | 50 μM |
|---|---|---|
| AR-001 | 23.7 | 245.7 |
| AR-002 | −1.5 | 1.3 |
| AR-003 | −1.1 | 3.9 |
| AR-004 | −0.7 | −0.8 |
| AR-005 | 0.2 | 250.8 |
| AR-006 | 169.6 | 288.6 |
| AR-007 | −1.1 | 12.0 |
| AR-008 | 0.4 | 2.6 |
| AR-009 | −1.2 | 14.6 |
| AR-010 | 2.1 | 239.5 |
| AR-011 | −0.4 | 42.8 |
| AR-012 | −0.6 | 6.4 |
| AR-013 | 0.8 | −0.4 |
| AR-014 | −0.9 | 25.1 |
| AR-015 | 28.6 | 219.0 |
| AR-016 | 3.7 | 100.6 |
| AR-017 | 0.6 | 0.9 |
| AR-018 | 1.0 | 0.0 |
| AR-019 | −0.3 | 0.0 |
| AR-020 | 0.3 | −0.7 |
| AR-021 | 1.8 | 0.1 |
| AR-022 | −0.5 | 0.0 |
| AR-023 | 1.7 | 47.8 |
| AR-024 | −0.7 | 0.1 |
| AR-025 | −1.6 | 0.4 |
| AR-026 | −0.6 | 0.0 |
| AR-027 | 0.2 | 1.1 |
| AR-028 | −0.3 | −1.1 |

### INDUSTRIAL APPLICABILITY

The novel compounds of the present invention have industrial applicability because they can shorten sleep latency and increase total sleep duration and thus they can be effectively used in a pharmaceutical composition for preventing or treating a sleep disorder, a health functional food composition for preventing or relieving a sleep disorder, a composition for improving sleep, a sleep aid or a method of treating a sleep disorder.

1.-12. (canceled)

13. A compound represented by [Chemical Formula 1] below, an isomer thereof, or a pharmaceutically acceptable salt thereof: 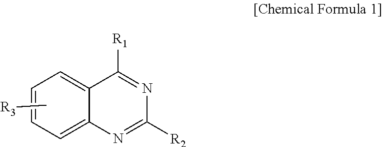
wherein R₁ is any one selected from the group consisting of 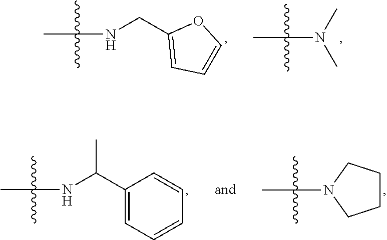
R² is any one selected from the group consisting of 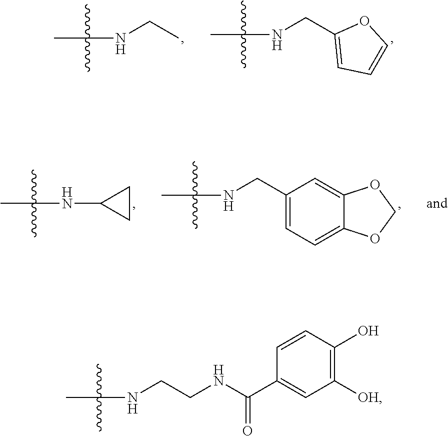
and
R₃ is one or more selected from the group consisting of hydrogen, hydroxy, and methoxy.

14. A pharmaceutical composition for treating a sleep disorder, comprising the compound, the isomer thereof, or the pharmaceutically acceptable salt thereof according to claim 13 and a pharmaceutically acceptable carrier.

15. A health functional food composition for relieving a sleep disorder, comprising the compound, the isomer thereof, or the pharmaceutically acceptable salt thereof of claim 13.

16. A method of treating a sleep disorder, comprising: administering the compound, the isomer thereof, or the pharmaceutically acceptable salt thereof according to claim 13 to a patient with a sleep disorder.

17. The method of claim 16, wherein the sleep disorder is one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.

18. A method of treating a sleep disorder, comprising: administering the health functional food composition according to claim 15 to a patient with a sleep disorder.

19. The method of claim 18, wherein the sleep disorder is one or more selected from the group consisting of sleep onset disorder, sound sleep disorder, awakening during sleep, early awakening, insomnia, nightmare, sleepwalking, narcolepsy, abnormal behavior during sleep, hypersomnia, sleep seizures, breathing-related sleep disorder, apnea, circadian rhythm sleep disorder, parasomnia, restless leg syndrome, and periodic limb movement disorder.
